SEQUENCE LISTING (7 sequences)

SEQ ID NO: 1 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 2 - protein, 139 aa, Artificial Sequence
  Synthetic polypeptide
MKHLWFFLLLVAAPRWVLSQVQLQESGPGLVKPSETLSLTCTVSGGSISIYYWSWIRQPPGKGLEWIGYVYYSGSTNYNPSLKSRVTISVDTSKNQFSLKLNSVTAADTAVYYCARGGYDFWSGYFDYWGQGTLVTVSS

SEQ ID NO: 3 - protein, 128 aa, Artificial Sequence
  Synthetic polypeptide
MEAPAQLLFLLLLWLPDTTGEIVMTQSPATLSVSPGERATLSCRASQSVDSNLAWYRQKPGQAPRLLIYGASTRATGIPARFSGSGSGTEFTLTISSLQSEDFAVYYCQQYINWPPITFGQGTRLEIK

SEQ ID NO: 4 - protein, 107 aa, Homo sapiens [sequence omitted]

SEQ ID NO: 5 - protein, 330 aa, Homo sapiens [sequence omitted]

SEQ ID NO: 6 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 7 - protein, 1408 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

(19) United States
(12) Patent Application Publication
(10) Pub. No.: US 2014/0234328 A1
(43) Pub. Date: Aug. 21, 2014

(54) USE OF C-MET PROTEIN FOR PREDICTING THE EFFICACY OF ANTI-HEPATOCYTE GROWTH FACTOR ("HGF") ANTIBODIES IN ESOPHAGEAL AND GASTRIC CANCER PATIENTS

(71) Applicants: Abraham Anderson, Sherman Oaks, CA (US); Kelly Oliner, Newbury Park, CA (US); Rui Tang, Thousand Oaks, CA (US); Elwyn Loh, Redwood City, CA (US); Sarita Dubey, Burlingame, CA (US)

(72) Inventors: Abraham Anderson, Sherman Oaks, CA (US); Kelly Oliner, Newbury Park, CA (US); Rui Tang, Thousand Oaks, CA (US); Elwyn Loh, Redwood City, CA (US); Sarita Dubey, Burlingame, CA (US)

(73) Assignee: AMGEN INC., THOUSAND OAKS, CA (US)

(21) Appl. No.: 14/343,328

(22) PCT Filed: Sep. 7, 2012
(86) PCT No.: PCT/US2012/054312

§ 371 Date: Mar. 6, 2014

Related U.S. Application Data
(60) Provisional application No. 61/533,097, filed on Sep. 9, 2011.
(60) Provisional application No. 61/533,089, filed on Sep. 9, 2011.

(51) Int. Cl. G01N 33/574; C07K 16/22

(52) U.S. Cl. CPC G01N 33/57446; C07K 16/22
USPC 424/142.1; 435/7.23

(57) ABSTRACT

The present invention relates to use of the human Met receptor (also known as “c-Met”) for predicting the efficacy of inhibitors of the HGF-Met pathway, and in particular, anti-HGF antibodies, in the treatment of esophageal and gastric cancer patients. The present invention also relates to methods and kits for predicting the usefulness of anti-HGF antibodies in the treatment of esophageal and gastric cancer.

### SEQUENCE LISTING

The instant application contains a. Sequence Listing which has been submitted in ASCII format via EFS-Web and is hereby incorporated by reference in its entirety. Said ASCII copy, created on Sep. 7, 2012, is named A-1671.txt and is 33,956 bytes in size.

### FIELD

The present invention relates to use of the human Met receptor (also known as “c-Met”) for predicting the efficacy of inhibitors of the HGF-Met pathway, and in particular, anti-HGF antibodies, in the treatment of esophageal and gastric cancer patients. The present invention also relates to methods and kits for predicting the usefulness of anti-HGF antibodies in the treatment of esophageal and gastric cancer.

### BACKGROUND

Esophageal and gastric cancers are among the most highly lethal types of cancer worldwide, with an annual incidence of approximately 1.5 million cases. The five-year relative survival rate for gastric cancer cases diagnosed in the United States has improved only from 16% to 24% over the last 30 years (Jemal, A, Siegel, R, Ward, E, et al. Cancer statistics, 2007. CA Cancer J Clin. 2007; 57:43-46), highlighting the need for more effective therapies. In addition, in Western countries, there has been a steady rise in adenocarcinomas of the gastric cardia and gastroesophageal junction, which has been associated with increased body mass index (Merry, A, Schouten, L, Goldbohm, A, et al. Body mass index, height and risk of adenocarcinoma of the esophagus and gastric cardia: a prospective cohort study. Gut. 2007,56:1503-1511). C-Met overexpression has been correlated with tumor depth of invasion, lymph node metastasis, stage, and peritoneal dissemination. In addition, c-Met overexpression has been correlated with shortened survival in patients with gastric cancer (Nakajima, M, Sawada, H, Yamada, Y, et al. The prognostic significance of amplification and overexpression of c-Met and c-erb B2 in human gastric carcinomas. Cancer. 1999; 85:1894-1902; and Taniguchi, K, Yonemura, Y, Nojima, N, et al. The relation between the growth patterns of gastric carcinoma and the expression of hepatocyte growth factor receptor (c-Met), autocrine motility factor receptor, and urokinase-type plasminogen activator receptor. Cancer. 1998; 82: 2112-2122). Moreover, elevated serum hepatocyte growth factor (HGF) levels at gastric cancer diagnosis have been correlated with stage of disease and have been shown to decrease following resection (Tanka, K, Miki, C, Wakuda, R, et al. Circulating level of hepatocyte growth factor as a useful marker in patients with early-stage gastric carcinoma. Scand J Gastroenterol. 2004; 39:754-760 and Han, S, Le, J, Kim, W, et. al. Significant correlation between serum level of hepatocyte growth factor and progression of gastric carcinoma. World J Surg. 1999; 23:1176-1180, and Beppu, K, Uchiyama, A, Morisaki, K, et al. Elevation of serum hepatocyte growth factor concentration in patients with gastric cancer is mediated by production from tumor tissue. Anticancer Res. 2000; 20:1263-1267). The majority (80% to 100%) of esophageal adenocarcinomas express c-Met by immunohistochemistry (Herrera, L, El-Hefnawy, T, Queiroz, P, et al. The HGF receptor c-Met is overexpressed in esophageal adenocarcinoma. Neoplasia. 2005; 7:75-84). Chemotherapy regimens in early trials of advanced gastric cancer often included 5-FU, an anthracycline, and methotrexate (eg, 5-FU, methotrexate, adriamycin, and leucovorin regimen [FAMTX]). In more recent trials cisplatin has been used in combination with 5-FU. The REAL1 trial demonstrated an overall survival (OS) advantage for treatment with epirubicin, cisplatin, and 5-FU (ECF) as compared to FAMTX (Webb, A. Cunningham, D, Scarffe, J, et al. Randomized trial comparing epirubicin, cisplatin, and fluorouracil versus fluorouracil, doxorubicin, and methotrexate in advanced esophagogastric cancer. J Clin Oncol. 1997; 15: 261-267). In general, the ECF combination has demonstrated response rates of 40-50%, time to tumor progression of approximately 5 to 7 months and median OS of 9 to 10 months. The use of docetaxel with cisplatin and 5-FU (DCF) has also shown activity in treatment of advanced gastric cancer.

Current treatments achieve modest improvements in survival, but there it is still a need to identify new and effective therapies. Therefore, it is desirable to identify and clinically validate markers that can be used to evaluate whether an individual diagnosed with esophageal and/or gastric cancer will respond to a therapeutic agent before commencing treatment with the agent.

### SUMMARY

One problem that can confront patients and health care professionals is the appropriate selection of a treatment regime for a patient, particularly when various treatment options are available, as is the case with gastric cancer, including but not limited to locally advanced or metastatic gastric or esophagogastric junction adenocarcinoma. Methods and reagents useful for informing appropriate treatment options using anti-HGF antibodies, and more particularly, rilotumumab, to treat patients diagnosed with gastric cancer, including but not limited to locally advanced or metastatic gastric or esophagogastric junction adenocarcinoma are described herein. The methods and reagents described herein are used to provide guidance as to which patients are likely to respond to treatment with anti-HGF antibodies, such as rilotumumab. Rilotumumab was evaluated in combination with the chemotherapy agents, epirubicin, cisplatin and capecitabine (“ECX”) in a multicenter, Phase 2, randomized, double-blind, placebo-controlled study as a treatment for gastric cancer, including but not limited to locally advanced or metastatic gastric or esophagogastric junction adenocarcinoma (Amgen Study Number 20060317 or the '317 Study). At the end of the 30 week dosing period, compared to placebo plus an ECX control, enhancements were shown in overall survival and in progression-free survival when rilotumumab was administered in combination with ECX.

Patient archival tumor specimens were collected in the '317 Study and analyzed to determine levels of biomarkers prior to treatment with rilotumumab. C-Met protein in tumor cells obtained from patients diagnosed with gastric cancer prior to treatment was identified as an independent predictive biomarker of clinical response. These results indicate that measuring c-Met protein levels in tumor cells obtained from patients diagnosed with gastric cancer is useful for predicting the patient's response to treatment with an anti-HGF antibody. Accordingly, in one embodiment of the invention, a method is described for predicting the efficacy of an anti-HGF antibody, comprising the step of determining a percentage of tumor cells having c-Met protein in a sample obtained from a patient diagnosed with gastric cancer, wherein a percentage of at least 1 percent of the tumor cells having c-Met predicts that administration of the anti-HGF antibody will treat the gastric cancer in the patient.

In addition, the disclosure provides methods of using c-Met protein levels as predictive biomarkers for determining whether or not a patient diagnosed with gastric cancer will respond to treatment with an anti-HGF antibody. Therefore, in another embodiment of the invention, a method is described of predicting whether a patient suffering from gastric cancer will respond to treatment with an anti-HGF antibody, comprising the step of determining a percentage of tumor cells having c-Met protein in a sample obtained from a patient diagnosed with gastric cancer, wherein a percentage of at least 1 percent of tumor cells having c-Met protein predicts that administration of the anti-HGF antibody will treat the gastric cancer in the patient.

In addition, the disclosure provides methods for using c-Met protein to screen for patients diagnosed with gastic cancer as being response to treatment with an anti-HGF antibody. Therefore in another embodiment of the invention, a method is described for screening for patients diagnosed with gastric cancer as being responsive to treatment with an anti-HGF antibody comprising the step of determining a percentage of tumor cells having c-Met protein present in a sample obtained from a patient diagnosed with gastric cancer, wherein a percentage of at least 1 percent of tumor cells having c-Met protein predicts that the patient with gastric cancer will be responsive to treatment with an anti-HGF antibody.

In some embodiments of these aspects of the invention, c-Met protein is measured in at least about 5 percent of the tumor cells. In some embodiments of these aspects of the invention, c-Met protein is measured in at least about 10 percent of the tumor cells. In some embodiments of these aspects of the invention, c-Met protein is measured in at least about 15 percent of the tumor cells. In some embodiments of these aspects of the invention, c-Met protein is measured in at least about 20 percent of the tumor cells. In some embodiments of these aspects of the invention, c-Met protein is measured in at least about 25 percent of the tumor cells. In some embodiments of these aspects of the invention, c-Met protein is measured in at least about 30 percent of the tumor cells. In some embodiments of these aspects of the invention, c-Met protein is measured in at least about 35 percent of the tumor cells. In some embodiments of these aspects of the invention, c-Met protein is measured in at least about 40 percent of the tumor cells. In some embodiments of these aspects of the invention, c-Met protein is measured in at least about 45 percent of the tumor cells. In some embodiments of these aspects of the invention, c-Met protein is measured in at least about 50 percent of the tumor cells. In some embodiments of these aspects of the invention, c-Met protein is measured in at least about 55 percent of the tumor cells. In some embodiments of these aspects of the invention, c-Met protein is measured in at least about 60 percent of the tumor cells. In some embodiments of these aspects of the invention, c-Met protein is measured in at least about 65 percent of the tumor cells. In some embodiments of these aspects of the invention, c-Met protein is measured in at least about 70 percent of the tumor cells. In some embodiments of these aspects of the invention, c-Met protein is measured in at least about 75 percent of the tumor cells. In some embodiments of these aspects of the invention, c-Met protein is measured in at least about 80 percent of the tumor cells. In some embodiments of these aspects of the invention, c-Met protein is measured in at least about 85 percent of the tumor cells. In some embodiments of these aspects of the invention, c-Met protein is measured in at least about 90 percent of the tumor cells.

In some embodiments of these aspects of the invention, the c-Met protein is measured in the cytoplasm of the tumor cells. In some embodiments of these aspects of the invention, the c-Met protein is measured in the membrane of the tumor cells. In some embodiments of these aspects of the invention, the c-Met protein is measured in the cytoplasm and the membrane of the tumor cells. In some embodiments of these aspects of the invention, the c-Met protein is a total measure of c-Met in the tumor cells, e.g., cytoplasm, membrane and other tumor cell organelles.

In another embodiment of the invention, a method is described for predicting the efficacy of an anti-HGF antibody, comprising the step of determining the maximum staining intensity of c-Met protein in tumor cells obtained from a patient diagnosed with gastric cancer, wherein a maximum staining intensity of at least 1 predicts that administration of the anti-HGF antibody will treat the gastric cancer in the patient when administered.

In still another embodiment of this aspect of the invention, a method is described for predicting whether a patient suffering from gastric cancer will respond to treatment with an anti-HGF antibody, comprising the step of determining the maximum staining intensity of c-Met protein in tumor cells obtained from a patient diagnosed with gastric cancer, wherein a maximum staining intensity of at least 1 predicts that administration of the anti-HGF antibody will treat the gastric cancer in the patient.

In addition, the disclosure provides methods for using c-Met protein to screen for patients diagnosed with gastric cancer as being response to treatment with an anti-HGF antibody. Accordingly, another embodiment of this invention is a method of screening for patients diagnosed with gastric cancer as being responsive to treatment with an anti-HGF antibody comprising the step of determining the maximum staining intensity of c-Met protein in tumor cells obtained from a patient diagnosed with gastric cancer, wherein a maximum staining intensity of at least 1 predicts that the patient with gastric cancer will be responsive to treatment with an anti-HGF antibody.

In some embodiments of these aspects of the invention, the maximum staining intensity is at least 2. In some embodiments of these aspects of the invention, the maximum staining intensity is at least 3. In some embodiments of these aspects of the invention, the c-Met protein is measured in the cytoplasm of the tumor cells.

In some embodiments of these aspects of the invention, the c-Met protein is measured in the membrane of the tumor cells. In some embodiments of these aspects of the invention, the c-Met protein is measured in the cytoplasm and the membrane of the tumor cells. In some embodiments of these aspects of the invention, the c-Met protein is a total measure of c-Met in the tumor cells, e.g., cytoplasm, membrane and other tumor cell organelles.

In another embodiment of the invention, a method is described for predicting the efficacy of an anti-HGF antibody, comprising the step of determining the H-score for c-Met protein in tumor cells obtained from a patient diagnosed with gastric cancer, wherein an H-score of at least 1 predicts that administration of the anti-HGF antibody will treat the gastric cancer in the patient.

In still another embodiment of this aspect of the invention, a method is described for predicting whether a patient suffering from gastric cancer will respond to treatment with an anti-HGF antibody, comprising the step of determining the H-score for c-Met protein in tumor cells obtained from a patient diagnosed with gastric cancer, wherein an H-score for c-Met protein of greater than 1 predicts that administration of the anti-HGF antibody will treat the gastric cancer in the patient.

In addition, the disclosure provides methods for using c-Met protein to screen for patients diagnosed with gastric cancer as being response to treatment with an anti-HGF antibody. Accordingly, another embodiment of this invention is a method of screening for patients diagnosed with gastric cancer as being responsive to treatment with an anti-HGF antibody comprising the step of determining the H-score for c-Met protein in tumor cells obtained from a patient diagnosed with gastric cancer, wherein an H-score for c-Met protein of greater than 1 predicts that administration of the anti-HGF antibody will treat the gastric cancer in the patient.

In some embodiments of these aspects of the invention, the H-score is greater than about 10. In some embodiments of these aspects of the invention, the H-score is greater than about 25. In some embodiments of these aspects of the invention, the H-score is greater than about 50. In some embodiments of these aspects of the invention, the H-score is greater than about 75. In some embodiments of these aspects of the invention, the H-score is greater than about 100. In some embodiments of these aspects of the invention, the H-score is greater than about 125. In some embodiments of these aspects of the invention, the H-score is greater than about 150. In some embodiments of these aspects of the invention, the H-score is greater than about 175. In some embodiments of these aspects of the invention, the H-score is greater than about 200. In some embodiments of these aspects of the invention, the H-score is greater than about 225. In some embodiments of these aspects of the invention, the H-score is greater than about 250. In some embodiments of these aspects of the invention, the H-score is greater than about 275. In some embodiments of these aspects of the invention, the H-score is greater than about 300.

In some embodiments of these aspects of the invention, the c-Met protein is measured in the cytoplasm of the tumor cells. In some embodiments of these aspects of the invention, the c-Met protein is measured in the membrane of the tumor cells. In some embodiments of these aspects of the invention, the c-Met protein is measured in the cytoplasm and the membrane of the tumor cells. In some embodiments of these aspects of the invention, the c-Met protein is a total measure of c-Met in the tumor cells, e.g., cytoplasm, membrane and other tumor cell organelles.

Patients that exhibit a level of c-Met protein in their tumor sample above the defined threshold value are better candidates for treatment with an anti-HGF antibody, such as rilotumumab, Therefore, in still another aspect, the present disclosure provides methods of treating patients diagnosed with gastric cancer and having c-Met protein in their tumor sample above the defined threshold value. In one aspect of this invention, a method of treating a patient diagnosed with gastric cancer is described, wherein a sample of tumor cells obtained from the patient diagnosed with gastric cancer has a percentage of at least 1 percent of the tumor cells having c-Met protein present, as measured in an in vitro assay, the method comprising the step of administering to a patient diagnosed with gastric cancer an anti-HGF antibody effective to provide a therapeutic benefit.

In some embodiments of these aspects of the invention, c-Met protein is measured in at least about 5 percent of the tumor cells. In some embodiments of these aspects of the invention, c-Met protein is measured in at least about 10 percent of the tumor cells. In some embodiments of these aspects of the invention, c-Met protein is measured in at least about 15 percent of the tumor cells. In some embodiments of these aspects of the invention, c-Met protein is measured in at least about 20 percent of the tumor cells. In some embodiments of these aspects of the invention, c-Met protein is measured in at least about 25 percent of the tumor cells. In some embodiments of these aspects of the invention, c-Met protein is measured in at least about 30 percent of the tumor cells. In some embodiments of these aspects of the invention, c-Met protein is measured in at least about 35 percent of the tumor cells. In some embodiments of these aspects of the invention, c-Met protein is measured in at least about 40 percent of the tumor cells. In some embodiments of these aspects of the invention, c-Met protein is measured in at least about 45 percent of the tumor cells. In some embodiments of these aspects of the invention, c-Met protein is measured in at least about 50 percent of the tumor cells. In some embodiments of these aspects of the invention, c-Met protein is measured in at least about 55 percent of the tumor cells. In some embodiments of these aspects of the invention, c-Met protein is measured in at least about 60 percent of the tumor cells. In some embodiments of these aspects of the invention, c-Met protein is measured in at least about 65 percent of the tumor cells. In some embodiments of these aspects of the invention, c-Met protein is measured in at least about 70 percent of the tumor cells. In some embodiments of these aspects of the invention, c-Met protein is measured in at least about 75 percent of the tumor cells. In some embodiments of these aspects of the invention, c-Met protein is measured in at least about 80 percent of the tumor cells. In some embodiments of these aspects of the invention, c-Met protein is measured in at least about 85 percent of the tumor cells. In some embodiments of these aspects of the invention, c-Met protein is measured in at least about 90 percent of the tumor cells.

In some embodiments of this aspect of the invention, the c-Met protein is measured in the cytoplasm of the tumor cells. In some embodiments of these aspects of the invention, the c-Met protein is measured in the membrane of the tumor cells. In some embodiments of these aspects of the invention, the c-Met protein is measured in the cytoplasm and the membrane of the tumor cells. In some embodiments of these aspects of the invention, the c-Met protein is a total measure of c-Met in the tumor cells, e.g., cytoplasm, membrane and other tumor cell organelles.

In another aspect of this invention, a method of treating a patient diagnosed with gastric cancer is described, wherein a sample of tumor cells obtained from the patient diagnosed with gastric cancer has a maximum staining intensity of c-Met protein in tumor cells of at least 1, as measured in an in vitro assay, the method comprising the step of administering to a patient diagnosed with gastric cancer an anti-HGF antibody effective to provide a therapeutic benefit.

In some embodiments of this aspect of the invention, the maximum staining intensity is at least 2. In some embodiments of these aspects of the invention, the maximum staining intensity is at least 3. In some embodiments of these aspects of the invention, the c-Met protein is measured in the cytoplasm of the tumor cells.

In some embodiments of these aspects of the invention, the c-Met protein is measured in the membrane of the tumor cells. In some embodiments of these aspects of the invention, the c-Met protein is measured in the cytoplasm and the membrane of the tumor cells. In some embodiments of these aspects of the invention, the c-Met protein is a total measure of c-Met in the tumor cells, e.g., cytoplasm, membrane and other tumor cell organelles.

In still another aspect of this embodiment of this invention, a method of treating a patient diagnosed with gastric cancer is described, wherein a sample of tumor cells obtained from the patient diagnosed with gastric cancer has an H-score for c-Met protein of at least 1, as measured in an in vitro assay, the method comprising the step of administering to a patient diagnosed with gastric cancer an anti-HGF antibody effective to provide a therapeutic benefit.

In some embodiments of these aspects of the invention, the H-score is greater than about 10. In some embodiments of these aspects of the invention, the H-score is greater than about 25. In some embodiments of these aspects of the invention, the H-score is greater than about 50. In some embodiments of these aspects of the invention, the H-score is greater than about 75. In some embodiments of these aspects of the invention, the H-score is greater than about 100. In some embodiments of these aspects of the invention, the H-score is greater than about 125. In some embodiments of these aspects of the invention, the H-score is greater than about 150. In some embodiments of these aspects of the invention, the H-score is greater than about 175. In some embodiments of these aspects of the invention, the H-score is greater than about 200.

In some embodiments of these aspects of the invention, the c-Met protein is measured in the cytoplasm of the tumor cells. In some embodiments of these aspects of the invention, the c-Met protein is measured in the membrane of the tumor cells. In some embodiments of these aspects of the invention, the c-Met protein is measured in the cytoplasm and the membrane of the tumor cells. In some embodiments of these aspects of the invention, the c-Met protein is a total measure of c-Met in the tumor cells, e.g., cytoplasm, membrane and other tumor cell organelles.

In some embodiments of these aspects of the invention, the c-Met protein is measured by an immunohistochemistry (IHC) assay.

In some embodiments of these aspects of the invention, the anti-HGF antibody specifically binds to the beta-subunit of the human HGF protein. In some embodiments of the invention the anti-HGF antibody is selected from the group consisting of rilotumumab, ficlatuzumab and TAK 701. In some embodiments of the invention, the anti-HGF antibody is rilotumumab.

In some embodiments of this aspect of the invention, rilotumumab is administered to a patient in need thereof at a dose of about 0.5 to about 30 milligrams per kilogram. In some embodiments of this aspect of the invention, rilotumumab is administered at a dose of about 7.5 to about 20 milligrams per kilogram. In some embodiments of this aspect of the invention, rilotumumab is administered at a dose of 5.0 mg/kg. In some embodiments of this aspect of the invention, rilotumumab is administered at a dose of 7.5 mg/kg. In some embodiments of this aspect of the invention, rilotumumab is administered at a dose of 10 mg/kg. In some embodiments of this aspect of the invention, rilotumumab is administered at a dose of 15 mg/kg. In some embodiments of this aspect of the invention, rilotumumab is administered at a dose of 20 mg/kg. In some embodiments of this aspect of the invention, rilotumumab is administered intravenously, subcutaneously, intramuscularly, intranasally, or transdermally. In some embodiments of this aspect of the invention, rilotumumab is administered at least weekly. In some embodiments of this aspect of the invention, is administered at least bi-weekly. In some embodiments of this aspect of the invention, rilotumumab is administered at least every three weeks. In some embodiments of this aspect of the invention, rilotumumab is administered at least monthly.

In some embodiments of the invention, at least one other therapeutic agent is administered with the anti-HGF antibody. In some embodiments of this aspect of the invention, the other therapeutic agent that is administered in addition to the anti-HGF antibody is a chemotherapy agent. In some embodiments of this aspect of the invention, the chemotherapy agent is selected from the group consisting of epirubicin, cisplatin, capecitabine, 5-FU, methotrexate, adriamycin, leucovorin, S1, oxaliplatin, methotrexate, irinotecan, docetaxel and trastuzumab. In some embodiments of this aspect of the invention the other therapeutic agents are epirubicin, cisplatin and capecitabine. In some embodiments of this aspect of the invention, the epirubicin is administered at a dose of about 50 mg/m², cisplatin is administered at a dose of about 60 mg/m², and capecitabine is administered at a dose of about 625 mg/m². In some embodiments of this aspect of the invention the other therapeutic agents include cisplatin and capecitabine. In some embodiments of this aspect of the invention, the cisplatin is administered at a dose of about 80 mg/m² on day 1, and capecitabine is administered at a dose of about 1000 mg/m² twice daily on days 1-14 (cycle length is 21 days).

In some embodiments of this invention, the gastric cancer is more specifically locally advanced gastric cancer. In some embodiments of this aspect of this invention, the gastric cancer is more specifically metastatic gastric cancer. In some embodiments of this aspect of the invention, the gastric cancer is more specifically esophageal adenocarcinoma. In some embodiments of this aspect of the invention, the gastric cancer is more specifically esophagogastric junction adenocarcinoma.

### BRIEF DESCRIPTION OF THE FIGURES

FIG. 1 is a schematic drawing of the Phase 2 Study design for Amgen Trial 20060317.

FIG. 2A is Kaplan-Meier survival curves showing progression-free survival of patients within the low and high c-Met expression subgroup in the rilotumumab treated arms combined and the low and high c-Met expression subgroup within the placebo arm, and FIG. 2B is Kaplan-Meier survival curves showing overall survival of the patients within the low and high c-Met expression subgroup in the rilotumumab treated arms combined and the low and high c-Met expression subgroup within the placebo arm. For both FIGS. 2A and 2B, IHC subgroup is defined as cytoplasmic percent positive cells >50% (High) vs. cytoplasmic percent positive <=50% (Low).

FIG. 3A is Kaplan-Meier survival curves showing progression-free survival of patients within the low and high c-Met expression subgroup in the rilotumumab treated arms combined and the low and high c-Met expression subgroup within the placebo arm, and FIG. 3B is Kaplan-Meier survival curves showing overall survival of patients within the low and high c-Met expression subgroup in the rilotumumab treated arms combined and the low and high c-Met expression subgroup within the placebo arm. For both FIGS. 3A and 3B, IHC subgroup is defined as cytoplasmic percent positive cells >10% (High) vs. cytoplasmic percent positive <=10% (Low).

FIG. 4A is a Kaplan-Meier survival curves showing progression-free survival of patients within the low and high c-Met expression subgroup in the rilotumumab treated arms combined and the low and high c-Met expression subgroup within the placebo arm, and FIG. 4B is a Kaplan-Meier survival curves showing overall survival of patients within the low and high c-Met expression subgroup in the rilotumumab treated arms combined and the low and high c-Met expression subgroup within the placebo arm. For both FIGS. 4A and 4B, IHC subgroup is defined as cytoplasmic percent positive cells >80% (High) vs. cytoplasmic percent positive <=80% (Low).

FIGS. 5A and 5B are a Forestplot summarizing a cox regression model evaluating the treatment effect in patients within the high/low c-Met IHC subgroups based on increasing (5 to 95) percentages of cytoplasmic positive samples.

FIG. 6A is a forestplot summarizing a cox regression model evaluating the treatment effect (Combined Rilotumumab Arms (“TRT”) versus Placebo Arm (“PBO”) in patients within the high/low c-Met IHC subgroups on Progression Free Survival (total staining); FIG. 6B is a forestplot summarizing a cox regression model evaluating the treatment effect (Combined TRT versus PBO) in patients within the high/low c-Met IHC subgroups on Progression Free Survival (cytoplasmic and membrane staining); FIG. 6C is a forestplot summarizing a cox regression model evaluating the treatment effect (Combined TRT versus PBO) in patients within the high/low c-Met IHC subgroups on Overall Survival (cytoplasmic and membrane staining; and FIG. 6D is a forestplot summarizing a cox regression model evaluating the treatment effect (Combined TRT versus PBO) in patients within the high/low c-Met IHC subgroups on Overall Survival (total staining).

FIG. 7A is the amino acid sequence of the human c-Met precursor protein, isoform B.

FIG. 7B is the amino acid sequence of the human c-Met precursor protein, isoform A.

FIG. 8 shows the amino acid sequences of the heavy chain variable region and the light chain variable region of rilotumumab. Antibody name, germ-line designation, and the sequence ID are indicated. The natural signal peptide sequence is underlined.

FIG. 9 is the amino acid sequences of the human kappa constant region, the human IgG1 constant region and the human IgG2 constant regions.

FIG. 10 is a scatter plot of Cytoplasmic H-Score versus Cytoplasmic Percent Positive.

### DETAILED DESCRIPTION

All references cited herein, including patents, patent applications, papers, textbooks, and the like, and the references cited therein, to the extent that they are not already, are hereby incorporated herein by reference in their entirety for any purpose. In the event that one or more of the documents incorporated by reference defines a term that contradicts that term's definition in the instant disclosure, this disclosure controls. The section headings used herein are for organizational purposes only and are not to be construed as limiting the subject matter described.

### DEFINITIONS

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclature utilized in connection with, and techniques of, cell and tissue culture, molecular biology, and protein and oligo- or polynucleotide chemistry described herein are those well known and commonly used in the art. Standard techniques are used for recombinant DNA, oligonucleotide synthesis, and tissue culture and transformation (e.g., electroporation, lipofection). Enzymatic reactions, purification and analytical techniques are performed according to the manufacturer's or service provider's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

Following standard convention, as used herein the terms “a” and “an” mean “one or more” unless context or explicit verbiage dictates otherwise.

In the instant disclosure, the term “or” means “and/or” unless stated otherwise. In the context of a multiple dependent claim, the use of “or” refers back to more than one preceding independent or dependent claim in the alternative only. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

“Native antibodies and immunoglobulins”, in certain instances, are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Chothia et al., J. Mol. Biol. 186:651 (1985: Novotny and Haber, Proc. Natl. Acad. Sci. U.S.A. 82:4592 (1985); Chothia et al., Nature 342:877-883 (1989)).

The term “antibody” refers to an immunoglobulin molecule that specifically binds to, or is immunologically reactive with, a particular antigen, and includes polyclonal, monoclonal, genetically engineered (e.g., rIgG) and otherwise modified forms of antibodies, including but not limited to chimeric antibodies, humanized antibodies, heteroconjugate antibodies (including e.g., bispecific antibodies), antigen binding fragments of antibodies, including e.g., Fab, Fab′, F(ab′)2, Fv, single-chain antibodies (“scFv”), Fd′ and Fd fragments and multimeric forms of antigen binding fragments, including e.g., diabodies, triabodies and tetrabodies. Moreover, unless otherwise indicated, the term “monoclonal antibody” (mAb) is meant to include both an intact antibody and an antigen binding fragment thereof which competes with the intact antibody for specific binding. “Antigen binding fragment thereof” refers to a portion or fragment of an intact antibody molecule, wherein the fragment retains the antigen-binding function. Binding fragments are produced by recombinant DNA techniques, or by enzymatic or chemical cleavage of intact antibodies such as by cleavage with papain. Methods for producing the various fragments from monoclonal antibodies are well known to those skilled in the art (see, e.g., Pluckthun, 1992, Immunol. Rev. 130:151-188). An antibody other than a “bispecific” or “bifunctional” antibody is understood to have each of its binding sites be identical. An antibody substantially inhibits adhesion of a receptor to a counter-receptor when an excess of antibody reduces the quantity of receptor bound to counter-receptor by at least about 20%, 40%, 60%, or 80%, and more usually greater than about 85%, 90%, 95%, 96%, 97%, 98%, or 99% (as measured in an in vitro competitive binding assay).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and terminal or internal amino acid sequencing by use of a spinning cup sequenator, or (2) to homogeneity by SDS-PAGE under reducing or nonreducing conditions using Coomassie blue or, preferably, silver stain. An isolated antibody includes the antibody in situ within recombinant cells since at least one component of the antibody's natural environment will not be present. Ordinarily, however, isolated antibody will be prepared by at least one purification step.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al. (1991). The constant domains are not involved directly in binding an antibody to an antigen, but exhibit various effector functions, such as participation of the antibody in antibody-dependent cellular toxicity.

“Fv” is the minimum antibody fragment which contains a complete antigen-recognition and binding site. In a two-chain Fv species, this region comprises a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity on the antibody. However, even a single variable domain (or half of an Fv comprising only three CDRs specific for an antigen) has the ability to recognize and bind antigen, although at a lower affinity than the entire binding site.

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-62 (L2), and 89-97 (L3) in the light chain variable domain and 31-55 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 ((H1), 53-55 (H2) and 96-101 (H3) in the heavy chain variable domain; Chothia and Lesk J. Mol. Biol 196:901-917 (1987)). “Framework Region” or “FR” residues are those variable domain residues other than the hypervariable region residues as herein defined.

The term “complementarity determining regions” or “CDRs,” when used herein, refers to parts of immunological receptors that make contact with a specific ligand and determine its specificity. The CDRs of immunological receptors are the most variable part of the receptor protein, giving receptors their diversity, and are carried on six loops at the distal end of the receptor's variable domains, three loops coming from each of the two variable domains of the receptor.

“Epitope” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The determination of whether two antibodies bind substantially to the same epitope is accomplished using methods known in the art, such as a competition assay. In conducting an antibody competition study between a control antibody (for example, rilotumumab) and any test antibody, one may first label the control antibody with a detectable label, such as, biotin, an enzyme, radioactive label, or fluorescent label to enable the subsequent identification. In such an assay, the intensity of bound label is measured in a sample containing the labeled control antibody and the intensity of bound label sample containing the labeled control antibody and the unlabeled test antibody is measured. If the unlabeled test antibody competes with the labeled antibody by binding to an overlapping epitope, the detected label intensity will be decreased relative to the binding in the sample containing only the labeled control antibody. Other methods of determining binding are known in the art.

The term “monoclonal antibody” refers to an antibody that is derived from a single clone, including any eukaryotic, prokaryotic, or phage clone, and not the method by which it is produced. Antibodies immunologically reactive with a particular antigen can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow and Lane, “Antibodies: A Laboratory Manual,” Cold Spring Harbor Laboratory Press, New York (1988); Hammerling et al., in: “Monoclonal Antibodies and T-Cell Hybridomas,” Elsevier, N.Y. (1981), pp. 563 681 (both of which are incorporated herein by reference in their entireties).

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Any of the anti-HGF antibodies described herein can be chimeric.

The term “humanized antibody” refers to an immunoglobulin comprising a human framework, at least one and preferably all complementarity determining regions (CDRs) from a non-human antibody, and in which any constant region present is substantially identical to a human immunoglobulin constant region, i.e., at least about 85%, at least about 90%, at least about 95%, and at least about 98% identical. Hence, all parts of a humanized immunoglobulin, except possibly the CDRs, are substantially identical to corresponding parts of one or more native human immunoglobulin sequences. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., Queen et al., U.S. Pat. Nos. 5,530,101; 5,585,089; 5,693,761; 5,693,762; 6,180,370 (each of which is incorporated by reference in its entirety). Antibodies can be humanized using a variety of techniques known in the art including, for example, CDR-grafting (EP 239,400; PCT publication WO 91/09967; U.S. Pat. Nos. 5,225,539; 5,530,101 and 5,585,089), veneering or resurfacing (EP 592,106; EP 519,596; Padlan, Mol. Immunol., 28:489 498 (1991); Studnicka et al., Prot. Eng. 7:805 814 (1994); Roguska et al., Proc. Natl. Acad. Sci. USA, 91:969 973 (1994), and chain shuffling (U.S. Pat. No. 5,565,332), all of which are hereby incorporated by reference in their entireties. Any of the anti-HGF antibodies described herein can be humanized antibodies, such as mouse humanized antibodies, etc.

An “anti-HGF antibody” is an antibody, or fragment thereof, that interferes with the binding between HGF and c-Met by specifically binding to and neutralizing hepatocyte growth factor (“HGF”), as shown with in vitro testing or by other means. In certain embodiments, an anti-HGF antibody specifically binds to any portion of HGF protein. In certain other embodiments, an anti-HGF antibody specifically binds the beta-subunit of HGF protein. In still other embodiments, an anti-HGF antibody specifically binds the N-terminal region of the beta-subunit of HGF protein.

The term “specifically binds” refers to the ability of a specific binding agent, such as an antibody, to bind to a target with greater affinity than it binds to a non-target. In certain embodiments, specific binding refers to binding for a target with an affinity that is at least 10, 50, 100, 250, 500, or 1000 times greater than the affinity for a non-target. In certain embodiments, affinity is determined by an affinity ELISA assay. In certain embodiments, affinity is determined by a BIAcore™ assay. In certain embodiments, affinity is determined by a kinetic method. In certain embodiments, affinity is determined by an equilibrium/solution method. In certain embodiments, an antibody is said to specifically bind an antigen when the dissociation constant between the antibody and one or more of its recognized epitopes is ≦1 M, preferably ≦100 nM and most preferably ≦10 nM.

Anti-HGF antibodies suitable for use in the methods described herein include monoclonal antibodies, chimeric antibodies, humanized antibodies, or fully human antibodies. Examples of anti-HGF antibodies capable of binding HGF include, but are not limited to, rilotumumab, and the humanized anti-HGF antibodies, ficlatuzumab and TAK701 (See, ficlatuzumab is a humanized monoclonal anti-HGF antibody, as described in WO 2007/143090 and U.S. Pat. No. 7,649,083 and TAK701 is a humanized monoclonal anti-HGF/SF antibody, as described in WO 2005/107800, WO 2007/115049, and U.S. Pat. No. 7,494,650 and U.S. Pat. No. 7,220,410, all of which are herein incorporated by reference in their entirety).

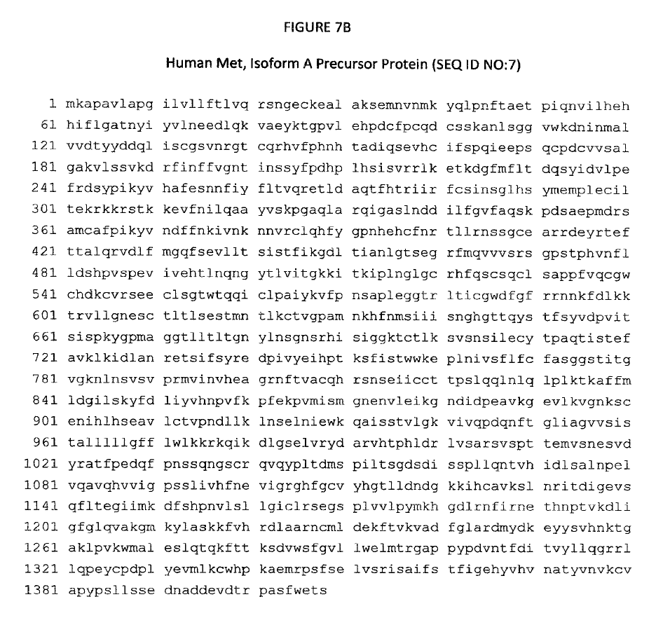

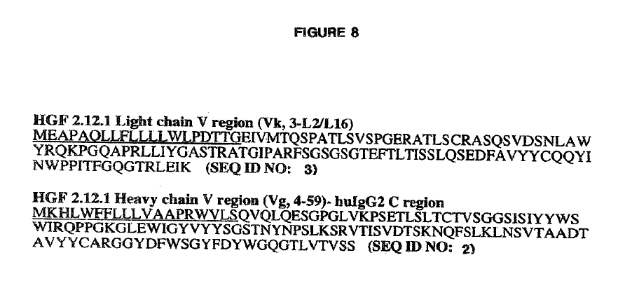

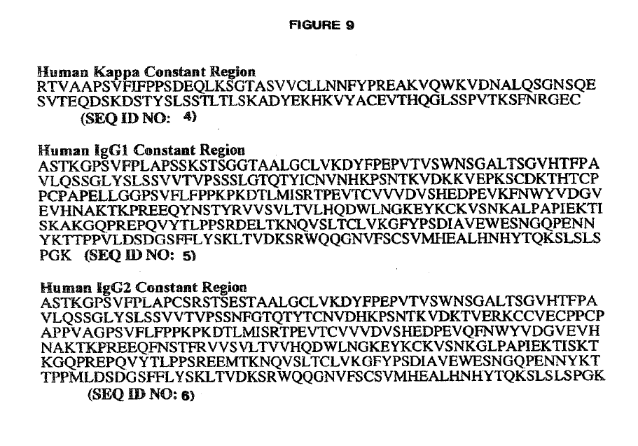

“Rilotumumab” refers to is an anti-HGF/SF antibody, as described in US Patent Publication No. 2005/0118643 and WO 2005/017107 which are herein incorporated by reference in its entirety, particularly in parts pertinent to rilotumumab, its structure and properties, methods for making and using it, and other related antibodies. Rilotumumab is identified in US 2005/0118643 and WO 2005/017107 as antibody 2.12.1. The amino acid sequence of the heavy chain variable region and the light chain variable region of rilotumumab is provided in FIG. 8 (SEQ ID NOs:2 and 3, respectively). In addition, the amino acid sequences of the human kappa constant region, the human IgG 1 constant region and the human IgG2 constant regions are provided in FIG. 9 (SEQ ID NOs:4-6, respectively). Also included in the definition of “rilotumumab” are antibodies that vary from rilotumumab, that that retain the ability to bind human HGF (e.g., “Bioequivalent”). Such variant antibodies comprise one or more additions, deletions or substitutions of amino acids when compared to the rilotumumab sequence, but exhibit biological activity that is essentially equivalent to that of rilotumumab, e.g., the blocking of the c-MET pathway.

Antibodies are considered “bioequivalent” if, for example the antibodies are pharmaceutical equivalents or pharmaceutical alternatives whose rate and extent of adsorption do not show a significant difference when administered at the same molar dose under similar experimental conditions, either single doses or multiple doses. Some antibodies will be considered equivalents or pharmaceutical alternatives if they are equivalent in the extent of their absorption but not in their rate of absorption and yet may be considered bioequivalent because such differences I the rate of absorption are intentional and are reflected in the labelling, are not essential to the attainment of effective body drug concentrations, e.g., chronic use, and are considered medically insignificant for the particular drug product studied. In one embodiment, two antibodies are bioequivalent if there are no clinically meaningful differences in their safety, purity, and/or potency. In one embodiment, two antibodies are bioequivalent if a patient can be switched one or more times between the reference product and the biological product without an expected increase in the risk of adverse effects, including a clinically significant change in immunogenicity, or diminished effectiveness, as compared to continued therapy without such switching. In one embodiment, two antibodies are bioequivalent if they both act by a common mechanism or mechanisms of action for the condition or conditions of use, to the extent that such mechanisms are known.

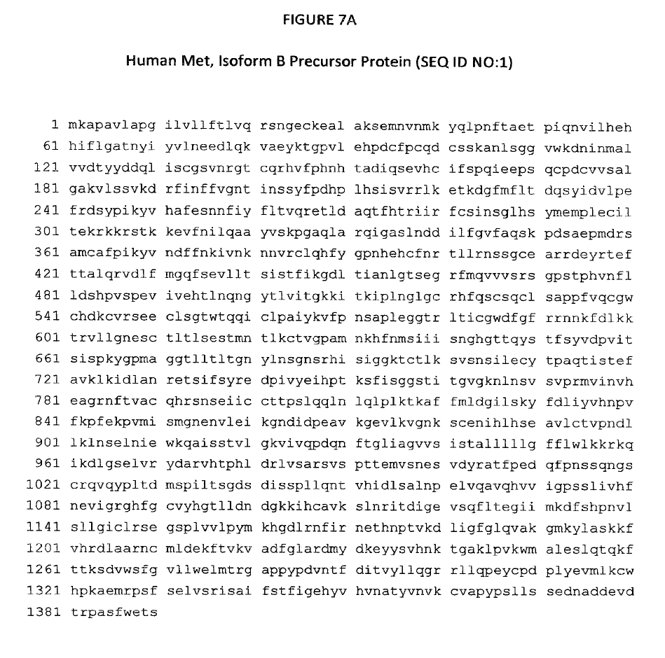

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time: (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody. Bioequivalent variants of rilotumumab may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. “C-Met protein” (also known as c-Met receptor or HGF receptor (“HGFr”)) refers to a high affinity tyrosine kinase receptor for HGF expressed on the cell surface of a variety of normal cells and primary solid tumours and in their metastases. C-Met protein is a disulfide-linked heterodimer made of 45 kDa alpha-subunits and 145 kDa beta-subunits. The amino acid sequences of the human MET precursor proteins, isoforms A and B are provided in FIG. 7 (Isoform A (SEQ ID NO:7; amino acids 1-1408) and Isoform B (SEQ ID NO: 1, amino acids 1-1390)). These sequences are further processed into a mature form. The extracellular domain of the mature protein for Isoform A is amino acids 25-950. The extracellular domain of the mature protein for Isoform B is amino acids 25-932.

“Tumour cells having c-Met” and “c-Met present in tumour cells” refers to the amount of c-Met protein in a patient sample, or for example, in tumor cells obtained from a patient diagnosed with gastric cancer. C-Met protein can be measured in a number of in vitro assays, known to those skilled in the art, including but not limited to inmmunohistochemistry (“IHC”), ELISA, Western and immunoprecipitation. C-Met protein can also be quantified or scored in a variety of ways known to those skilled in the art, and include, but are not limited to the following: the percentage of tumour cells having c-Met in the cytoplasm of the cells; the percentage of tumour cells having c-Met in the membrane of the cells: the percentage of tumour cells having c-Met anywhere in the cells (total, e.g., cytoplasm, membrane, other organelles, etc.); the maximum cytoplasmic or membrane or total staining intensity of c-Met protein in a patient sample (e.g., tumour cells); and/or the cytoplasmic or membrane or total H-score for c-Met protein in a patient sample (e.g., tumour cells).

As used herein, the term “maximum staining intensity of c-Met protein” means the maximum intensity level of staining of c-Met protein in a patient sample, or for example, in tumor cells obtained from a patient diagnosed with gastric cancer. In one embodiment, the maximum intensity level of staining of c-Met protein in the cytoplasm of the patient tumor cells is measured. In another embodiment, the maximum intensity level of staining of c-Met protein in the membrane of the patient tumor cells is measured. In still another embodiment, the maximum intensity level of staining of c-Met protein anywhere in the patient tumor cells is measured (i.e., total, e.g., cytoplasm, membrane, other organelles, etc.). In a specific context, four possible levels of staining intensity exist: 0 (unstained), I+(weak staining), 2+(moderate staining), and 3+(strong staining), as determined by a laboratory defined test (“LDT”) described herein c-Met.

As used herein, the term “H-score for c-Met protein” means the level of c-Met protein in a sample, or for example in tumor cells obtained from a patient diagnosed with gastric cancer. In one embodiment, the H-score for c-Met protein is a measurement of the c-Met protein in the cytoplasm of the patient tumor cells. In another embodiment, the H-score for c-Met protein is a measurement of the c-Met protein in the membrane of the patient tumor cells.

In still another embodiment, the H-score is a measurement of the c-Met protein anywhere in the patient tumor cells (i.e., total, e.g., cytoplasm, membrane, other organelles, etc.). In a specific context, H-scores can be calculated based on the summation of the product of percent of cells stained at each intensity using the following equation: (3×% cells staining 3+)+(2×% cells staining 2+)+(1×% cells staining 1+), as determined by a LDT described herein. The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., 3H, 14C, 15N, 35S, 90Y, 99Tc, 111In, 125I, 131I), fluorescent labels (e.g., FITC, rhodamine, lanthanide phosphors), enzymatic labels (e.g., horseradish peroxidase, β-galactosidase, luciferase, alkaline phosphatase), chemiluminescent groups, biotinyl groups, and predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

The terms, “therapeutic agent” or “pharmaceutical agent” or drug” as used herein refer to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, 96, 97, 98, or 99%. Most preferably, the object species is purified to essential homogeneity (contaminant species cannot be detected in the composition by conventional detection methods) wherein the composition consists essentially of a single macromolecular species.

The term “patient” includes human subjects.

The terms “mammal” and “animal” for purposes of treatment refers to any animal classified as a mammal, including humans, domestic and farm animals, and zoo, sports, or pet animals, such as dogs, horses, cats, cows, etc. Preferably, the mammal is human.

“Sample” refers to a sample from a human, animal, or to a research sample, e.g., cell, tissue, organ, fluid, gas, aerosol, slurry, colloid, or coagulated material. The “sample” may be tested in vivo, e.g., without the removal from the human or animal, or it may be tested in vitro. The sample may be tested after processing, e.g., by histological methods. “Sample” also refers, e.g., to a cell comprising a fluid or tissue sample or a cell separated from a fluid or tissue sample. “Sample” may also refer to a cell, tissue, organ, or fluid that is freshly taken from a human or animal, or to a cell, tissue, organ or fluid that is processed or stored. The term “disease state” refers to a physiological state of a cell or of a whole mammal in which an interruption, cessation, or disorder of cellular or body functions, systems, or organs has occurred.

The terms “treat” or “treatment” refers to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment, or receiving only partial treatment. Those in need of treatment include those already with the condition or disorder as well as those prone to have the condition or disorder or those in which the condition or disorder is to be prevented.

The term “responsive” as used herein means that a patient or tumor shows a complete response or a partial response after administering an agent, according to RECIST (Response Evaluation Criteria in Solid Tumors). The term “nonresponsive” as used herein means that a patient or tumor shows stable disease or progressive disease after administering an agent, according to RECIST. RECIST is described, e.g., in Therasse et al., February 2000, “New Guidelines to Evaluate the Response to Treatment in Solid Tumors,” J. Natl. Cancer Inst. 92(3): 205-216, which is incorporated by reference herein in its entirety. Exemplary agents include anti-HGF antibodies, including but not limited to rilotumumab.

“Therapeutically effective amount” of a therapeutic agent or therapeutic agents is defined as an amount of the therapeutic agent or therapeutic agents that is sufficient to show meaningful patient benefit, i.e., to cause a decrease in, amelioration of, or prevention of the symptoms of the condition being treated. For purposes of this invention, meaningful patient benefit includes, but is not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable.

A “disorder is any condition that would benefit from one or more treatments. This includes chronic and acute disorders or disease including those pathological conditions which predispose the mammal to the disorder in question. Non-limiting examples of disorders to be treated herein include benign and malignant tumors, leukemias, and lymphoid malignancies, in particular stomach or gastric, colon or esophageal cancer. In a certain embodiment of this invention, the disorder to be treated in accordance with the present invention is a malignant tumor, such as gastric tumors, renal cell carcinoma (RCC), esophageal tumors, and carcinoma-derived cell lines.

“Gastric cancer” (also known as stomach cancer) is a disease in which the cells forming the inner lining of the stomach become abnormal and start to divide uncontrollably forming a mass called a tumor. The term “gastric cancer” as used herein includes, but is not limited to, locally advanced and metastatic gastric and esophagogastric junction adenocarcinoma. In “combined therapy,” patients are treated with an anti-HGF antibody and at least one other therapeutic agent. In certain embodiments, patients are treated with an anti-HGF antibody and at least one other chemotherapeutic agent. In certain embodiments, the anti-HGF antibody is rilotumumab and the other therapeutic agent comprises epirubicin, cisplatin and capecitabine. Protocol designs will address effectiveness as assessed by reduction in tumor mass as well as the ability to reduce usual doses of standard chemotherapy. These dosage reductions will allow additional and/or prolonged therapy by reducing dose-related toxicity of the chemotherapeutic agent.

### General Considerations

Gastric cancer is the second most common cause of cancer death world-wide. HGF and c-Met expression are implicated in gastric cancer. Rilotumumab is a fully human antibody that specifically binds to HGF and interferes with the binding of HGF to c-Met.

Provided herewith are methods directed to a patient having gastric cancer. As defined above, gastric cancer includes, but is not limited to locally advanced and metastatic gastric and esophagogastric junction adenocarcinoma. Often times, gastric cancer is routinely identified by practitioners in the field of oncology, such as physicians, medical oncologists, histopathologists and oncologic clinicians.

The data provided in the below example show that the administration of an anti-HGF antibody and at least one other therapeutic agent leads to progression-free survival as well as overall survival of the treated disease. More specifically, the data show that the administration of an anti-HGF antibody, rilotumumab, and ECX leads to progression-free survival as well as overall survival of the treated disease. The actual effect appears to be correlated with the expression level of c-Met protein in the surface of the malignant cells to be treated. Therefore, any patient diagnosed with gastric cancer, having certain levels of c-Met protein in their tumor cells may benefit from the disclosed methods. There is no requirement as to the stage of the patient's tumor; the tumor can be at any stage of growth, for example T2, T3 or T4. The tumor can also exist at any stage of the nodal stage, for example NO, N1. N2a, N2b, N2c or N3. Further, the tumor can be staged as such by any system, for example the AJCC system or the TNM staging system.

Responsiveness or nonresponsiveness to treatment with an anti-HGF antibody and at least one other therapeutic agent can be determined using any established criteria. In a specific example, responsiveness or nonresponsiveness can be determined using the widely adopted RECIST (Response Evaluation Criteria in Solid Tumors) criteria. See, e.g., Therasse et al., (2000) J. Natl. Cancer inst. 92(3): 205-216, which is incorporated by reference herein for any purpose. Complete response and partial response according to RECIST are both considered to be responsive to treatment with an anti-HGF antibody and at least one additional therapeutic agent. Stable disease and progressive disease are both considered to be nonresponsive to treatment with an anti-HGF antibody and at least one additional therapeutic agent.

All of the disclosed methods can be supplemented as desired. For example, the disclosed methods can be supplemented by adjusting the therapy of a patient having gastric cancer based on an evaluation of the results of the method. In one embodiment, a patient not receiving therapy comprising an anti-HGF antibody and at least one other therapeutic agent can be placed on such a regimen, based on the determination of the c-Met protein level in the patient's sample of tumor cells.

### Method of Predicting Whether a Patient Having Gastric or Esophagogastric Junction Adenocarcinoma Will Benefit from Treatment Comprising an Anti-HGF Antibody and at Least One Other Therapeutic Agent

C-Met has been identified as a potential prognostic marker in gastric cancer (see, e.g., Drebber et al., (2008) Oncol Rep. June; 19(6):1477-83). Until the instant disclosure, however, c-Met has not been ascribed a predictive role, particularly in the area of anti-HGF antibody-based therapies. Accordingly, in one aspect of the instant disclosure, a method of predicting whether a patient having gastric cancer will benefit from treatment comprising an anti-HGF antibody. In one embodiment the method comprises determining the level of c-Met protein in a sample from a patient diagnosed with gastric cancer, wherein if the patient's sample has a certain level of c-Met protein, the patient is predicted to benefit from treatment with an anti-HGF antibody. In another embodiment, the method comprises determining the level of c-Met protein in a sample from a patient diagnosed with gastric cancer, wherein if the patient's sample has a certain level of c-Met protein, the patient is predicted to benefit from treatment with an anti-HGF antibody, such as rilotumumab. In another embodiment, the method comprises determining the level of c-Met protein in a sample from a patient diagnosed with gastric cancer, wherein if the patient's sample has a certain level of c-Met protein, the patient is predicted to benefit from treatment with an anti-HGF antibody, such as rilotumumab when, administered in combination with a chemotherapy regimen. In another embodiment, the method comprises determining the level of c-Met protein in a sample from a patient diagnosed with gastric cancer, wherein if the patient's sample has a certain level of c-Met protein, the patient is predicted to benefit from treatment with an anti-HGF antibody, such as rilotumumab when, administered in combination with a chemotherapy regimen, such as ECX, cisplatnin and capecitabine (“CX”) epirubicin-cisplatin-5-FY (“ECF”), epirubicin-oxaliplatin-capecitabine (“EOX”), or S1 and cisplatin. In yet another embodiment, the method comprises determining the level of c-Met protein in a sample from a patient diagnosed with gastric cancer, wherein if the patient's sample has a certain level of c-Met protein, the patient is predicted to benefit from treatment with an anti-HGF antibody, such as rilotumumab, and at least one other therapeutic agent, such as ECX, or CX.

Initially the c-Met protein level is determined from a sample of tumor cells obtained from a patient diagnosed with gastric cancer. In order to make the determination, any convenient method can be employed. For example, techniques as varied as IHC, FISH, qPCR or a mass spectrometry-based approach can be employed. Most often, it will be desirable to obtain a sample of the patient's tumor and perform the determination in an in vitro setting.

In one specific embodiment the c-Met protein level of a tumor sample obtained from a patient diagnosed with gastric cancer can be readily determined using any commercially available kit or a service provider. For example, an in vitro diagnostic kit from Leica Microsystems (Hepatocyte Growth Factor Receptor (c-MT)(clone 8F11)) or Ventana Medical Systems (CONFIRM c-MET (Total) (catalog number 790-4430)) can be employed to determine the c-Met protein levels. Alternatively, a sample of the patient's tumor can be supplied to a provider, such as Mosaic Laboratories in Lake Forest, Calif., which can perform an in vitro assay or a Laboratory Defined Test (“LDT”) such as an IHC assay described in Example 1, and report the results. In yet another example, an anti-c-Met antibody can be generated and used as a component of an in vitro assay, such as an IHC procedure.

The determination of c-Met protein levels in a sample from a patient diagnosed with gastric cancer can be made on the basis of standard scoring guidelines. The guidelines can be quantitative, semi-quantitative or qualitative. In one example, IHC can be used to evaluate on a semi-quantitative scale, the percentage of tumor cells having c-Met protein, and the percentage of cancer cells staining at each of the following four levels can be recorded as: 0 (unstained), 1+(weak staining), 2+(moderate staining) and 3+(strong staining). Using this methodology, a patient sample having a percentage of at least about 1 percent of tumor cells having c-Met protein predicts that administration of the anti-HGF antibody will treat the gastric cancer in the patient. In another specific embodiment, a patient sample having a percentage of at least about 5, at least about 10, at least about 15, at least about 20, at least about 25, at least about 30, at least about 35, at least about 40; at least about 45, at least about 50, at least about 55, at least about 60, at least about 65, at least about 70, at least about 75, at least about 80, at least about 85, at least about 90, at least about 95, at least about 98 percent of tumor cells having c-Met protein predicts that administration of the anti-HGF antibody will treat the gastric cancer in the patient. In another specific embodiment, a patient sample having a percentage of at least about 1, at least about 5, at least about 10, at least about 15, at least about 20, at least about 25, at least about 30, at least about 35, at least about 40; at least about 45, at least about 50, at least about 55, at least about 60, at least about 65, at least about 70, at least about 75, at least about 80, at least about 85, at least about 90, at least about 95, at least about 98 percent of tumor cells having c-Met protein predicts that administration of the anti-HGF antibody, rilotumumab, will treat the gastric cancer in the patient. In another specific embodiment, a patient sample having a percentage of at least about 1, at least about 5, at least about 10, at least about 15, at least about 20, at least about 25, at least about 30, at least about 35, at least about 40; at least about 45, at least about 50, at least about 55, at least about 60, at least about 65, at least about 70, at least about 75, at least about 80, at least about 85, at least about 90, at least about 95, at least about 98 percent of tumor cells having c-Met protein predicts that the anti-HGF antibody, rilotumumab, when administered in combination with at least one other therapeutic agent, will treat the gastric cancer in the patient. In another specific embodiment, a patient sample having a percentage of at least about 1, at least about 5, at least about 10, at least about 15, at least about 20, at least about 25, at least about 30, at least about 35, at least about 40; at least about 45, at least about 50, at least about 55, at least about 60, at least about 65, at least about 70, at least about 75, at least about 80, at least about 85, at least about 90, at least about 95, at least about 98 percent of tumor cells having c-Met protein predicts that the anti-HGF antibody, rilotumumab, when administered in combination with at least one chemotherapy regiment such as ECX, CX, ECF, EOX, or S1 and cisplatin, will treat the gastric cancer in the patient. In one specific embodiment, the c-Met protein present in the cytoplasm of the tumor cells is measured. In another embodiment, the c-Met protein in the membrane of the tumor cells is measured. In still another embodiment, the total c-Met protein in the tumor cells, including but not limited to the c-Met protein in the cytoplasm, the membrane and other organelles of the tumor cells.

In another specific embodiment, the maximum staining intensity of c-Met protein in tumor cells from a patient diagnosed with gastric cancer is measured using IHC. In one embodiment, a patient sample having a maximum staining intensity of at least 1 predicts that administration of the anti-HGF antibody will treat the gastric cancer in the patient. In another embodiment, a patient sample having a maximum staining intensity of at least 2 predicts that administration of the anti-HGF antibody will treat the gastric cancer in the patient. In yet another embodiment, a patient sample having a maximum staining intensity of at least 3 predicts that administration of the anti-HGF antibody will treat the gastric cancer in the patient when administered. In another embodiment, a patient sample having a maximum staining intensity of at least 1, at least 2, at least 3, predicts that administration of the anti-HGF antibody will treat the gastric cancer in the patient when administered in combination with at least one other therapeutic agent, such as a chemotherapy regimen as described herein. The c-Met assay can be evaluated on a semi-quantitative scale, and the percentage of cancer cells staining at each of the following four levels is recorded: 0 (unstained), 1+(weak staining), 2+(moderate staining) and 3+(strong staining).

In yet another specific embodiment, an H-score for c-Met protein in tumor cells obtained from a patient diagnosed with gastric cancer is determined. An H-score can be calculated based on the summation of the product of percent of cells stained at each intensity using the following equation: (3×% cells staining at 3+)+(2×% cells staining at 2+)+(1×% cells staining at 1+). In one embodiment, an H-score of at least 1 predicts that administration of the anti-HGF antibody will treat the gastric cancer in the patient. In another embodiment, an H-score of at least 10 predicts that administration of the anti-HGF antibody will treat the gastric cancer in the patient. In another embodiment, an H-score of at least 20 predicts that administration of the anti-HGF antibody will treat the gastric cancer in the patient. In another embodiment, an H-score of at least 30 predicts that administration of the anti-HGF antibody will treat the gastric cancer in the patient. In another embodiment, an H-score of at least 40 predicts that administration of the anti-HGF antibody will treat the gastric cancer in the patient. In another embodiment, an H-score of at least 50 predicts that administration of the anti-HGF antibody will treat the gastric cancer in the patient. In another embodiment, an H-score of at least 75 predicts that administration of the anti-HGF antibody will treat the gastric cancer in the patient. In one embodiment, an H-score of at least 100 predicts that administration of the anti-HGF antibody will treat the gastric cancer in the patient. In another embodiment, an H-score of at least 125 predicts that administration of the anti-HGF antibody will treat the gastric cancer in the patient. In another embodiment, an H-score of at least 150 predicts that administration of the anti-HGF antibody will treat the gastric cancer in the patient. In another embodiment, an H-score of at least 175 predicts that administration of the anti-HGF antibody will treat the gastric cancer in the patient. In one embodiment, an H-score of at least 200 predicts that administration of the anti-HGF antibody will treat the gastric cancer in the. In another embodiment, an H-score of at least 225 predicts that administration of the anti-HGF antibody will treat the gastric cancer in the patient. In another embodiment, an H-score of at least 250 predicts that administration of the anti-HGF antibody will treat the gastric cancer in the patient. In another embodiment, an H-score of at least 275 predicts that administration of the anti-HGF antibody will treat the gastric cancer in the patient. In still another embodiment, an H-score of at least 1, at least 10, at least 20, at least 30, at least 40, at least 50, at least 75, at least 100, at least 125, at least 150, at least 175, at least 200, at least 225, at least 250, at least 275 predicts that administration of an anti-HGF antibody will treat the gastric cancer in the patient when administered in combination with at least one other therapeutic agent, such as a chemotherapy agent.

As demonstrated by the data presented in the Example, patients diagnosed with gastric cancer whose tumor samples had certain levels of c-Met protein and who received therapy comprising an anti-HGF antibody showed an enhancement in progression-free survival and in overall survival. Thus, if the patient diagnosed with gastric cancer has certain levels of c-Met protein in their tumor sample, as described above, the patient is predicted to benefit from treatment with an anti-HGF antibody, such as rilotumumab. Moreover, if the patient diagnosed with gastric cancer has certain levels of c-Met protein in their tumor sample, as described above, the patient is predicted to benefit from treatment with an anti-HGF antibody, such as rilotumumab, and at least one other therapeutic agent, such as ECX or CX.

### Method of Screening a Population of Patients Having Locally Advanced or Metastatic Gastric or Esophagogastric Junction Adenocarcinoma

As demonstrated by the data presented in the Example, patients having gastric cancer, including but not limited to locally advanced or metastatic gastric or esophagogastric junction adenocarcinoma, whose tumors have certain levels of c-Met protein will benefit from a therapy comprising an anti-HGF antibody. Accordingly, it can be desirable to screen or identify or stratify such patients for treatment with an anti-HGF antibody using c-Met protein levels as an indicator. Thus, in another aspect of the current disclosure, a method of screening or stratifying a population of patients having a gastric cancer, including but not limited to locally advanced or metastatic gastric or esophageal adenocarcinoma or esophagogastric junction adenocarcinoma, into groups that will benefit more from therapy comprising an anti-HGF antibody is provided. In a specific embodiment, the method comprises determining the level of c-Met protein in a sample from a patient diagnosed with gastric cancer, wherein if the patient's sample has a certain level of c-Met protein, the patient is predicted to benefit from treatment with an anti-HGF antibody, such as rilotumumab. In another specific embodiment, the method comprises determining the level of c-Met protein in a sample from a patient diagnosed with gastric cancer, wherein if the patient's sample has a certain level of c-Met protein, the patient is predicted to benefit from treatment with an anti-HGF antibody, such as rilotumumab, when administered with at least one other therapeutic agent, such as ECX, CX, ECF, EOX, or S1 and cisplatin.

When performing the method, the patient's c-Met protein level is determined. As is the case will all of the disclosed methods, in order to make the determination, any convenient method can be employed. For example, techniques as varied as IHC, FISH, qPCR or a mass spectrometry-based approach can be employed. Most often, it will be desirable to obtain a sample of the patient's tumor and perform the determination in an in vitro setting.

In one specific embodiment the c-Met protein level of a tumor sample obtained from a patient diagnosed with gastric cancer can be readily determined using any commercially available kit or a service provider. For example, an in vitro diagnostic kit from Leica Microsystems (Hepatocyte Growth Factor Receptor (c-MT)(clone 8F11)) or Ventana Medical Systems (CONFIRM c-MET (Total) (catalog number 790-4430)) can be employed to determine the c-Met protein levels. Alternatively, a sample of the patient's tumor can be supplied to a provider, such as Mosaic Laboratories in Lake Forest, Calif., which can perform an in vitro assay or a Laboratory Defined Test (“LDT”) such as an IHC assay described in Example 1, and report the results. In yet another example, an anti-c-Met antibody can be generated and used as a component of an in vitro assay, such as an IHC procedure.

The determination of c-Met protein levels in a sample from a patient diagnosed with gastric cancer can be made on the basis of scoring guidelines. The guidelines can be quantitative, semi-quantitative or qualitative. In one example, IHC can be used to evaluate on a semi-quantitative scale, the percentage of tumor cells having c-Met protein, and the percentage of cancer cells staining at each of the following four levels can be recorded as: 0 (unstained), 1+(weak staining), 2+(moderate staining) and 3+(strong staining). Using this methodology, a patient sample having a percentage of at least about 1 percent of tumor cells having c-Met protein predicts that administration of the anti-HGF antibody will treat the gastric cancer in the patient. In another specific embodiment, a patient sample having a percentage of at least about 5, at least about 10, at least about 15, at least about 20, at least about 25, at least about 30, at least about 35, at least about 40; at least about 45, at least about 50, at least about 55, at least about 60, at least about 65, at least about 70, at least about 75, at least about 80, at least about 85, at least about 90, at least about 95, at least about 98 percent of tumor cells having c-Met protein predicts that administration of the anti-HGF antibody will treat the gastric cancer in the patient. In another specific embodiment, a patient sample having a percentage of at least about 1, at least about 5, at least about 10, at least about 15, at least about 20, at least about 25, at least about 30, at least about 35, at least about 40; at least about 45, at least about 50, at least about 55, at least about 60, at least about 65, at least about 70, at least about 75, at least about 80, at least about 85, at least about 90, at least about 95, at least about 98 percent of tumor cells having c-Met protein predicts that administration of the anti-HGF antibody, rilotumumab, and at least one other therapeutic agent will treat the gastric cancer in the patient. In another specific embodiment, a patient sample having a percentage of at least about 1, at least about 5, at least about 10, at least about 15, at least about 20, at least about 25, at least about 30, at least about 35, at least about 40; at least about 45, at least about 50, at least about 55, at least about 60, at least about 65, at least about 70, at least about 75, at least about 80, at least about 85, at least about 90, at least about 95, at least about 98 percent of tumor cells having c-Met protein predicts that the anti-HGF antibody, rilotumumab, when administered in combination with at least one chemotherapy regiment such as ECX, CX, ECF, EOX, or S1 and cisplatin, will treat the gastric cancer in the patient. In one specific embodiment, the c-Met protein present in the cytoplasm of the tumor cells is measured. In another embodiment, the c-Met protein in the membrane of the tumor cells is measured. In still another embodiment, the total c-Met protein in the tumor cells, including but not limited to the c-Met protein in the cytoplasm, the membrane and other organelles of the tumor cells.

In another specific embodiment, the maximum staining intensity of c-Met protein in tumor cells from a patient diagnosed with gastric cancer is measured using IHC. In one embodiment, a patient sample having a maximum staining intensity of at least 1 predicts that administration of the anti-HGF antibody will treat the gastric cancer in the patient. In another embodiment, a patient sample having a maximum staining intensity of at least 2 predicts that administration of the anti-HGF antibody will treat the gastric cancer in the patient. In yet another embodiment, a patient sample having a maximum staining intensity of at least 3 predicts that administration of the anti-HGF antibody will treat the gastric cancer in the patient. In another embodiment, a patient sample having a maximum staining intensity of at least 1, at least 2, at least 3, predicts that administration of the anti-HGF antibody will treat the gastric cancer in the patient when administered in combination with at least one other therapeutic agent, such as a chemotherapy regimen as described herein. The c-Met assay can be evaluated on a semi-quantitative scale, and the percentage of cancer cells staining at each of the following four levels is recorded: 0 (unstained), 1+(weak staining), 2+(moderate staining) and 3+ (strong staining).

In yet another specific embodiment, an H-score for c-Met protein in tumor cells obtained from a patient diagnosed with gastric cancer is determined. An H-score can be calculated based on the summation of the product of percent of cells stained at each intensity using the following equation: (3×% cells staining at 3+)+(2×% cells staining at 2+)+(1×% cells staining at 1+). In one embodiment, an H-score of at least 1 predicts that administration of the anti-HGF antibody will treat the gastric cancer in the patient. In another embodiment, an H-score of at least 10 predicts that administration of the anti-HGF antibody will treat the gastric cancer in the patient. In another embodiment, an H-score of at least 20 predicts that administration of the anti-HGF antibody will treat the gastric cancer in the patient. In another embodiment, an H-score of at least 30 predicts that administration of the anti-HGF antibody will treat the gastric cancer in the patient. In another embodiment, an H-score of at least 40 predicts that administration of the anti-HGF antibody will treat the gastric cancer in the patient. In another embodiment, an H-score of at least 50 predicts that administration of the anti-HGF antibody will treat the gastric cancer in the patient. In another embodiment, an H-score of at least 75 predicts that administration of the anti-HGF antibody will treat the gastric cancer in the patient. In one embodiment, an H-score of at least 100 predicts that administration of the anti-HGF antibody will treat the gastric cancer in the patient. In another embodiment, an H-score of at least 125 predicts that administration of the anti-HGF antibody will treat the gastric cancer in the patient. In another embodiment, an H-score of at least 150 predicts that administration of the anti-HGF antibody will treat the gastric cancer in the patient. In another embodiment, an H-score of at least 175 predicts that administration of the anti-HGF antibody will treat the gastric cancer in the patient. In one embodiment, an H-score of at least 200 predicts that administration of the anti-HGF antibody will treat the gastric cancer in the patient. In another embodiment, an H-score of at least 225 predicts that administration of the anti-HGF antibody will treat the gastric cancer in the patient. In another embodiment, an H-score of at least 250 predicts that administration of the anti-HGF antibody will treat the gastric cancer in the patient. In another embodiment, an H-score of at least 275 predicts that administration of the anti-HGF antibody will treat the gastric cancer in the patient. In still another embodiment, an H-score of at least 1, at least 10, at least 20, at least 30, at least 40, at least 50, at least 75, at least 100, at least 125, at least 150, at least 175, at least 200, at least 225, at least 250, at least 275, predicts that administration of an anti-HGF antibody will treat the gastric cancer in the patient when administered in combination with at least one other therapeutic agent, such as a chemotherapy agent, or chemotherapy agents (e.g., ECX, CX).

As demonstrated by the data presented in the Example, patients diagnosed with gastric cancer whose tumor samples had certain levels of c-Met protein and who received therapy comprising an anti-HGF antibody, showed an enhancement in progression-free survival and in overall survival. More specifically, the data in the Example demonstrated that patients diagnosed with gastric cancer whose tumor samples had certain levels of c-Met protein and who received therapy comprising rilotumumab in addition to the chemotherapy regimen, ECX, showed an enhancement in progression-free survival and in overall survival. Thus, if a patient diagnosed with gastric cancer has certain levels of c-Met protein in their tumor sample, as described above, the patient is predicted to benefit from treatment with an anti-HGF antibody, such as rilotumumab. Moreover, if the patient diagnosed with gastric cancer has certain levels of c-Met protein in their tumor sample, as described above, the patient is predicted to benefit from treatment with an anti-HGF antibody, such as rilotumumab, and at least one other therapeutic agent, such as ECX, or CX.

Continuing, patients whose tumors have the c-Met protein levels identified above are selected for treatment with a therapy comprising an anti-HGF antibody. These patients are expected to benefit more than patients who have lower c-Met protein levels than identified above in a therapy comprising an anti-HGF antibody. By screening for or stratifying a group of patients having gastric cancer with tumors that have the above-identified c-Met protein levels, a medical professional will be able to tailor a therapy to the patient's particular needs and enhances the likelihood that the patient will respond positively.

### Method of Treating a Patient Having Locally Advanced or Metastatic Gastric or Esophagogastric Junction Adenocarcinoma

As described herein and in the Example, it has been determined that patients having gastric cancer, including but not limited to locally advanced or metastatic gastric or esophagogastric junction adenocarcinoma, whose tumors have certain levels of c-Met protein will exhibit an enhancement in overall survival when treated with an anti-HGF antibody. Moreover, it has been determined that patients having gastric cancer, including but not limited to locally advanced or metastatic gastric or esophageal adenocarcinoma or esophagogastric junction adenocarcinoma, whose tumors have certain levels of c-Met protein will exhibit an enhancement in overall survival when treated with an anti-HGF antibody, such as rilotumumab, and at least one other therapeutic agent, such as ECX, CX, ECF, EOX, or S1 and cisplatin. Accordingly, a method of treating such patients is provided. In one embodiment of a method of treating a patient having locally advanced or metastatic gastric or esophageal adenocarcinoma, or esophagogastric junction adenocarcinoma comprises determining the c-Met protein level in a patient's tumor sample. As is the case with all of the disclosed methods, in order to make the determination, any convenient method can be employed. For example, techniques as varied as IHC, FISH, qPCR or a mass spectrometry-based approach can be employed. Most often, it will be desirable to obtain a sample of the patient's tumor and perform the determination in an in vitro setting.

In one specific embodiment the c-Met protein level of a tumor sample obtained from a patient diagnosed with gastric cancer can be readily determined using any commercially available kit or a service provider. For example, an in vitro diagnostic kit from Leica Microsystems (Hepatocyte Growth Factor Receptor (c-MT)(clone 8F11)) or Ventana Medical Systems (CONFIRM c-MET (Total) (catalog number 790-4430)) can be employed to determine the c-Met protein levels. Alternatively, a sample of the patient's tumor can be supplied to a provider, such as Mosaic Laboratories in Lake Forest, Calif., which can perform an in vitro assay or a Laboratory Defined Test (“LDT”) such as an IHC assay described in Example 1, and report the results. In yet another example, an anti-c-Met antibody can be generated and used as a component of an in vitro assay, such as an IHC procedure.

The determination of c-Met protein levels in a sample from a patient diagnosed with gastric cancer can be made on the basis of scoring guidelines. The guidelines can be quantitative, semi-quantitative or qualitative. In one example, IHC can be used to evaluate on a semi-quantitative scale, the percentage of tumor cells having c-Met protein, and the percentage of cancer cells staining at each of the following four levels can be recorded as: 0 (unstained), 1+(weak staining), 2+(moderate staining) and 3+(strong staining). Using this methodology, a patient sample having a percentage of at least about 1 percent of tumor cells having c-Met protein predicts that administration of the anti-HGF antibody will treat the gastric cancer in the patient. In another specific embodiment, a patient sample having a percentage of at least about 5, at least about 10, at least about 15, at least about 20, at least about 25, at least about 30, at least about 35, at least about 40; at least about 45, at least about 50, at least about 55, at least about 60, at least about 65, at least about 70, at least about 75, at least about 80, at least about 85, at least about 90, at least about 95, at least about 98 percent of tumor cells having c-Met protein predicts that administration of the anti-HGF antibody, In yet another specific embodiment, a patient sample having a percentage of at least about 1, at least about 5, at least about 10, at least about 15, at least about 20, at least about 25, at least about 30, at least about 35, at least about 40; at least about 45, at least about 50, at least about 55, at least about 60, at least about 65, at least about 70, at least about 75, at least about 80, at least about 85, at least about 90, at least about 95, at least about 98 percent of tumor cells having c-Met protein predicts that administration of the anti-HGF antibody and at least one therapeutic agent will treat the gastric cancer in the patient. In yet another specific embodiment, a patient sample having a percentage of at least about 1, at least about 5, at least about 10, at least about 15, at least about 20, at least about 25, at least about 30, at least about 35, at least about 40; at least about 45, at least about 50, at least about 55, at least about 60, at least about 65, at least about 70, at least about 75, at least about 80, at least about 85, at least about 90, at least about 95, at least about 98 percent of tumor cells having c-Met protein predicts that administration of the anti-HGF antibody, rilotumumab, and at least one therapeutic agent, such as a chemotherapy regimen (ECX, CX, ECF, EOX or S1 and cisplatin), will treat the gastric cancer in the patient. In another embodiment, the c-Met protein present in the cytoplasm of the tumor cells is measured. In another embodiment, the c-Met protein in the membrane of the tumor cells is measured. In still another embodiment, the total c-Met protein in the tumor cells, including but not limited to the c-Met protein in the cytoplasm, the membrane and other organelles of the tumor cells.

In another specific embodiment, the maximum staining intensity of c-Met protein in tumor cells from a patient diagnosed with gastric cancer is measured using IHC. In one embodiment, a patient sample having a maximum staining intensity of at least 1 predicts that administration of the anti-HGF antibody will treat the gastric cancer in the patient. In another embodiment, a patient sample having a maximum staining intensity of at least 2 predicts that administration of the anti-HGF antibody will treat the gastric cancer in the patient. In yet another embodiment, a patient sample having a maximum staining intensity of at least 3 predicts that administration of the anti-HGF antibody will treat the gastric cancer in the patient. In another embodiment, a patient sample having a maximum staining intensity of at least 1, at least 2, at least 3, predicts that administration of the anti-HGF antibody will treat the gastric cancer in the patient when administered in combination with at least one other therapeutic agent, such as a chemotherapy regimen as described herein. The c-Met assay was evaluated on a semi-quantitative scale, and the percentage of cancer cells staining at each of the following four levels was recorded: 0 (unstained), 1+(weak staining), 2+(moderate staining) and 3+(strong staining).

In yet another specific embodiment, an H-score for c-Met protein in tumor cells obtained from a patient diagnosed with gastric cancer is determined. An H-score can be calculated based on the summation of the product of percent of cells stained at each intensity using the following equation: (3×% cells staining at 3+)+(2×% cells staining at 2+)+(1×% cells staining at 1+). In one embodiment, an H-score of at least 1 predicts that administration of the anti-HGF antibody will treat the gastric cancer in the patient. In another embodiment, an H-score of at least 10 predicts that administration of the anti-HGF antibody will treat the gastric cancer in the patient. In another embodiment, an H-score of at least 20 predicts that administration of the anti-HGF antibody will treat the gastric cancer in the patient. In another embodiment, an H-score of at least 30 predicts that administration of the anti-HGF antibody will treat the gastric cancer in the patient. In another embodiment, an H-score of at least 40 predicts that administration of the anti-HGF antibody will treat the gastric cancer in the patient. In another embodiment, an H-score of at least 50 predicts that administration of the anti-HGF antibody will treat the gastric cancer in the patient. In another embodiment, an H-score of at least 75 predicts that administration of the anti-HGF antibody will treat the gastric cancer in the patient. In one embodiment, an H-score of at least 100 predicts that administration of the anti-HGF antibody will treat the gastric cancer in the patient. In another embodiment, an H-score of at least 125 predicts that administration of the anti-HGF antibody will treat the gastric cancer in the patient. In another embodiment, an H-score of at least 150 predicts that administration of the anti-HGF antibody will treat the gastric cancer in the patient. In another embodiment, an H-score of at least 175 predicts that administration of the anti-HGF antibody will treat the gastric cancer in the patient. In one embodiment, an H-score of at least 200 predicts that administration of the anti-HGF antibody will treat the gastric cancer in the patient. In another embodiment, an H-score of at least 225 predicts that administration of the anti-HGF antibody will treat the gastric cancer in the patient. In another embodiment, an H-score of at least 250 predicts that administration of the anti-HGF antibody will treat the gastric cancer in the patient. In another embodiment, an H-score of at least 275 predicts that administration of the anti-HGF antibody will treat the gastric cancer in the patient. In still another embodiment, an H-score of at least 1, at least 10, at least 20, at least 30, at least 40, at least 50, at least 75, at least 100, at least 125, at least 150, at least 175, at least 200, at least 225, at least 250, at least 275, predicts that administration of an anti-HGF antibody will treat the gastric cancer in the patient when administered in combination with at least one other therapeutic agent, such as a chemotherapy agent or chemotherapy agents (e.g., ECX, CX, etc.).

As demonstrated by the data presented in the Example, patients diagnosed with gastric cancer whose tumor samples had certain levels of c-Met protein and who received therapy comprising an anti-HGF antibody showed an enhancement in progression-free survival and in overall survival. Moreover, the data demonstrated that patients diagnosed with gastric cancer whose tumor samples had certain levels of c-Met protein and who received therapy comprising an anti-HGF antibody and at least one other therapeutic agent showed an enhancement in progression-free survival and in overall survival. Thus, if the patient diagnosed with gastric cancer has certain levels of c-Met protein in their tumor sample, as described above, the patient is predicted to benefit from treatment with an anti-HGF antibody, such as rilotumumab. Moreover, if the patient diagnosed with gastric cancer has certain levels of c-Met protein in their tumor sample, as described above, the patient is predicted to benefit from treatment with an anti-HGF antibody, such as rilotumumab and at least one other therapeutic agent, such as the chemotherapy agents, epirubicin, cisplatin and capecitabine.

Continuing with the method, if the patient's tumor has the above-identified c-Met protein level, the patient is administered an anti-HGF antibody. In certain embodiments, methods are provided of treating or preventing gastric cancer comprising administering a therapeutically effective amount of an anti-HGF-Met antibody and at least one other therapeutic agent. In certain embodiments, methods are provided of treating or preventing gastric cancer comprising administering a therapeutically effective amount of an anti-HGF-Met antibody and at least two other therapeutic agents. In certain embodiments, methods are provided of treating or preventing gastric cancer comprising administering a therapeutically effective amount of an anti-HGF-Met antibody and at least three other therapeutic agents. As shown in the data presented herein, patients having tumors that have the c-Met protein levels described above show enhancements in overall survival and in progression-free survival when administered an anti-HGF antibody, such as rilotumumab, and at least one therapeutic agent, such as a chemotherapy agent. In certain aspects of these embodiments, the chemotherapy agent is delivered as part of a chemotherapy regimen, such as ECX, CX, ECF, EOX or S1 and cisplatin. By performing the disclosed method, the medical professional can provide a more efficacious treatment regimen to patients suffering from this condition.

In certain embodiments, the administration of a therapeutically effective amount of an anti-HGF antibody and at least one other therapeutic comprises administering an anti-HGF antibody and at least one other therapeutic agent concurrently. In certain embodiments, the administration of a therapeutically effective amount of an anti-HGF antibody and at least one other therapeutic agent comprises administering an anti-HGF antibody prior to at least one other therapeutic agent. In certain embodiments, the administration of a therapeutically effective amount of an anti-HGF antibody and at least one other therapeutic agent comprises administering an anti-HGF antibody subsequent to at least one other therapeutic agent.

In certain embodiments, an anti-HGF antibody is rilotumumab, ficlatuzumab, and/or TAK 701. In one embodiment, an anti-HGF antibody is rilotumumab.

Therapeutic agents, include, but are not limited to, at least one other cancer therapy agent. Exemplary cancer therapy agents include, but are not limited to, chemotherapy and radiation therapy. Exemplary chemotherapy agents include, but are not limited to antineoplastic agents. Antineoplastic agents include, but are not limited to, antibiotic-type agents, alklylating agents, antimetabolite agents, hormonal agents, immunological agents, interferon-type agents, and miscellaneous agents.

In certain embodiments, an antineoplastic agent is an antimetabolite agent. Antimetabolite antineoplastic agents include, but are not limited to: 5-FU, fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrill Dow DDFC, dezaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, 5-fluorouracil, N-(2′-furanidyl)-5-fluorouracil, Daiichi Seiyaku FO-152, folinic acid, isopropyl pyrrolizine, leucovorin, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, thioguanine, tiazofurin. Erbamont TIF, trimetrexate, tyrosine kinase inhibitors, Taiho UFT and uricytin.

In certain embodiments, an antineoplastic agent is an aklylating-type agent. Alkylating-type antineoplastic agents include, but are not limited to: Shionogi 254-S, aldo-phosphamide analogues, altretamine, anaxirone, Boehringer Mannheim BBR-2207, bestrabucil, budotitane, Wakunaga CA-102, carboplatin, carmustine, Chinoin-139, Chinoin-153, chlorambucil, cisplatin, S1, cyclophosphamide, American Cyanamid CL-286558, Sanofi CY-233, cyplatate, Degussa D-19-384, Sumimoto DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsul-fam, ifosfamide, iproplatin, lomustine, mafosfamide, mitolactol, Nippon Kayaku NK-121. NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromus-tine, Tanabe Seiyaku TA-077, tauromustine, temozolomide, teroxirone, tetraplatin and trimelamol.

In certain embodiments, an antineoplastic agent is an antibiotic-type antineoplastic agent. Suitable antibiotic-type antineoplastic agents include, but are not limited to: Taiho 4181-A, aclarubicin, actinomycin D, actinoplanone, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, adriamycin, doxorubicin-fibrinogen, elsamicin-A, epirubicin, erbstatin, esorubicin, esperamicin-A1, esperamicin-Alb, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindanycin A, Tobishi RA-I, rapamycin, rhizoxin, rodorubicin, sibanomicin, siwenmycin, Sumitomo SM-5887, Snow Brand SN-706. Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024 and zorubicin.

Additional anti-neoplastic agent include, but are not limited to: tubulin interacting agents, topoisomerase II inhibitors, topoisomerase I inhibitors and hormonal agents, selected from but not limited to the group consisting of α-carotene, α-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluron, benzotript, Ipsen-Beaufour BIM-23015, bisantrene, Bristol-Myers BMY-40481, Vestar boron-10, bromofosfamide, Wellcome BW-502, Wellcome BW-773, caracemide, carmethizole hydrochloride, Ajinomoto CDAF, chlorsulfaquinoxalone, Chemes CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Yakult Honsha CPT-11, crisnatol, curaderm, cytochalasin B, cytarabine, cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, docetaxel elliprabin, elliptinium acetate, Tsumura EPMTC, the epothilones, ergotamine, etoposide, etretinate, fenretinide. Fujisawa FR-57704, gallium nitrate, genkwadaphnin, Chugai GLA-43, Glaxo GR-63178, grifolan NMF-5N, hexadecylphosphocholine, Green Cross HO-221, homoharringtonine, hydroxyurea, BTG ICRF-187, ilmofosine, isoglutamine, isotretinoin. Otsuka JI-36, Ramot K-477, Otsuak K-76COONa, Kureha Chemical K-AM, MECT Corp KI-8110, American Cyanamid L-623, leukoregulin, lonidamine, Lundbeck LU-23-112, Lilly LY-186641, NCI (US) MAP, marycin, Merrill Dow MDL-27048, Medco MEDR-340, merbarone, merocyanlne derivatives, methylanilinoacridine, Molecular Genetics MGI-136, minactivin, mitonafide, mitoquidone mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, ocreotide, Ono ONO-112, oquizanocine, Akzo Org-10172, paclitaxel, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, Sapporo Breweries RBS, restrictin-P, retelliptine, retinoic acid, Rhone-Poulenc RP-49532, Rhone-Poulenc RP-56976, SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, topotecan. Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, withanolides and Yamanouchi YM-534.

Additional anti-neoplastic agents include, but are not limited to: acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, BAM 002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburicase, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; AE 941 (Aeterna), ambamustine, antisense oligonucleotide, bcl-2 (Genta), APC 8015 (Dendreon), cetuximab, decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techniclone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

In certain embodiments, the therapeutic agent (or therapeutic agents) are selected from the group consisting of epirubicin, cisplatin, capecitabine, 5-FU, oxaliplatin, S1, irinotecan, docetaxel, trastuzumab, methotrexate, adriamycin, and leucovorin. In certain embodiments, the therapeutic agent is S1. In certain embodiments, the therapeutic agent is cisplatin. In certain embodiments, the therapeutic agent is capecitabine. In certain embodiments, the therapeutic agent is irinotecan. In certain embodiments, the therapeutic agent is epirubicin. In certain embodiments, the therapeutic agent is adriamycin. In certain embodiments, the therapeutic agent is oxaliplatin. In certain embodiments, the therapeutic agent is methotrexate. In certain embodiments, the therapeutic agent is docetaxel. In certain embodiments, the therapeutic agent is 5-FU. In certain embodiments, the therapeutic agent is trastuzumab. In certain embodiments, the therapeutic agents are cisplatin, and capecitabine. In certain embodiments, the therapeutic agents are epirubicin, cisplatin, and capecitabine. In certain embodiments, the therapeutic agents are epirubicin, cisplatin, and 5-FU. In certain embodiments, the therapeutic agents are epirubicin, oxaliplatin, and capecitabine.

In certain embodiments, in view of the condition and the desired level of treatment, two, three, or more therapeutic agents in addition to an anti-HGF antibody may be administered.

In certain embodiments, such agents may be provided together by inclusion in the same formulation. In certain embodiments, such agents may be formulated separately and provided together by inclusion in a treatment kit. In certain embodiments, such agents may be provided separately.

It is understood that the response by individual patients to the aforementioned medications or combination therapies may vary, and an appropriate efficacious combination of drugs for each patient may be determined by his or her physician.

In certain embodiments, the effective amount of an anti-HGF antibody to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which an anti-HGF antibody is being used, the route of administration, and the size (body weight, height, body surface and/or organ size) and/or condition (the age, physical condition, and/or general health) of the patient. In certain embodiments, the clinician will consider the severity and history of the disease for which an anti-HGF antibody is being used. In certain embodiments, the clinician may titer the dosage and modify the route of administration to obtain the optimal therapeutic effect.

In certain embodiments, a therapeutically effective dose of an anti-HGF antibody comprises an amount that ranges from about 0.01 mg/kg to about 500 mg/kg, from about 0.01 mg/kg to about 50 mg/kg, from about 0.5 mg/kg to about 30 mg/kg, or from about 7.5 to 20 mg/kg, or about 7.5 mg/kg, or about 10 mg/kg, or about 15 mg/kg, or about 20 mg/kg.

In certain embodiments, a therapeutically effective dose of an anti-HGF antibody comprises an amount of an antibody to HGF that ranges from about 0.5 mg/kg to about 30 mg/kg, administered weekly; about 2 mg/kg to about 20 mg/kg, administered weekly; about 5 mg/kg, administered weekly, or about 7.5 mg/kg, or about 10 mg/kg, or about 15 mg/kg, or about 20 mg/kg administered weekly; or about 0.5 mg/kg to about 20 mg/kg, administered every two weeks; about 3 mg/kg to about 20 mg/kg, administered every two weeks; about 10 mg/kg, administered every two weeks, or about 7.5 mg/kg, or about 10 mg/kg, or about 15 mg/kg, or about 20 mg/kg administered every two weeks: or about 7.5 mg/kg to about 30 mg/kg, about 10 mg/kg to about 20 mg/kg; or about 7.5 mg/kg, or about 10 mg/kg, or about 15 mg/kg, or about 20 mg/kg administered every three weeks; or about 10 mg/kg to about 30 mg/kg; or, or about 7.5 mg/kg, or about 10 mg/kg, or about 15 mg/kg, or about 20 mg/kg administered weekly administered every four weeks.

In certain embodiments, at least one other therapeutic agent is administered in combination with the anti-HGF antibody. As in the case of the anti-HGF antibody, the effective amount of the other therapeutic agent to be employed will depend, for example, upon the therapeutic context and objectives. One skilled in the art will appreciate that the appropriate dosage levels for treatment, according to certain embodiments, will thus vary depending, in part, upon the molecule delivered, the indication for which the additional therapeutic agent is being used, the route of administration, and the size (body weight, height, body surface and/or organ size) and/or condition (the age, physical condition, and/or general health) of the patient. In certain embodiments, the clinician will consider the severity and history of the disease for which an other therapeutic agent is being used. In certain embodiments, the clinician may titer the dosage and modify the route of administration to obtain the optimal therapeutic effect. The therapeutically effective dose of the additional therapeutic agent typically ranges from about 0.1 mg/m² to about 2400 mg/m², administered daily; or about 0.1 mg/m² to about 2400 mg/m², administered weekly; or from about 0.1 mg/m² to about 2400 mg/m², administered every two weeks; or about 0.1 mg/m² to about 2400 mg/m² administered every three weeks; or about 0.1 mg/m², to about 2400 mg/m², administered every four weeks.

In certain embodiments, the therapeutic agent comprises an amount of at least one chemotherapy agent selected from the group consisting of epirubicin, cisplatin, S1, capecitabine, leucovorin, 5-FU, oxaliplatin, irinotecan, docetaxel, trastuzumab, methotrexate, and adriamycin. In certain embodiments, the therapeutic agent is S1 that is administered at a dose of about 100 mg/m², administered daily for 21 days every 4 wks; or a dose of about 80 mg/m², administered daily for 28 days every 6 wks; or a dose of about 80 mg/m², administered daily for 14 days, every 3 wks; or a dose of about 80 mg/m², administered daily for 21 days every 5 wks.

In certain embodiments, the therapeutic agent is cisplatin that is administered at a dose of about 60 mg/m² to about 80 mg/m² once every three weeks; or at a dose of about 60 mg/m² to about 100 mg/m² once every four weeks.

In certain embodiments, the therapeutic agent is capecitabine that is administered at a dose of about 2000 mg/m², daily for 14 days every three weeks; or at a dose of about 1250 mg/m², daily.

In certain embodiments, the therapeutic agent is irinotecan administered at a dose of about 80 mg/m², weekly for six weeks; or 70 mg/m² once every two weeks for six weeks; or at a dose of about 180 mg/m² every two weeks.

In certain embodiments, the therapeutic agent is epirubicin that is administered at a dose of about 50 mg/m², once every three weeks; or at a dose of about 120 mg/m², once every four weeks.

In certain embodiments, the therapeutic agent is adriamycin that is administered at a dose of about 30 mg/m, once every four weeks; or at a dose of about 40 mg/m², once every five weeks.

In certain embodiments, the therapeutic agent is oxaliplatin that is administered at a dose of about 130 mg/m², once every three weeks.

In certain embodiments, the therapeutic agent is methotrexate that is administered at a dose of about 1500 mg/m², once every four weeks.

In certain embodiments, the therapeutic agent is docetaxel that is administered at a dose of about 30 mg/m², weekly; or at a dose of about 45 mg/m², once every two weeks; or at a dose of about 75 mg/m², once every three weeks.

In certain embodiments, the therapeutic agent is 5-FU that is administered at a dose of about 200 mg/m²: daily; or at a dose of about 1500 mg/m², daily for three days, every four weeks; or at a dose of about 1000 mg/m², daily for five days, every four weeks; or at a dose of about 800 mg/m², daily for five days, every three weeks; or at a dose of about 2400 mg/m², daily for two days, every two weeks.

In certain embodiments, the therapeutic agent is trastuzumab that is administered at a dose of about 8 mg/kg, once, followed by a dose of about 6 mg/kg every three weeks.

In certain embodiments, the therapeutic agent is S1 that is administered at a dose of about 100 mg/m², daily for 21 days, and cisplatin that is administered at a dose of about 75 mg/m² once every four weeks.

In some embodiments of this aspect of the invention the other therapeutic agents include cisplatin and capecitabine. In some embodiments of this aspect of the invention, the cisplatin is administered at a dose of about 80 mg/m² on day 1, and capecitabine is administered at a dose of about 1000 mg/m² twice daily on days 1-14 (cycle length is 21 days).

In certain embodiments, the therapeutic agent is epirubicin that is administered at a dose of about 50 mg/m², once every three weeks, and cisplatin that is administered at a dose of about 60 mg/m², once every three weeks, and 5-FU that is administered at a dose of about 200 mg/m², daily.

In certain embodiments, the therapeutic agent is epirubicin that is administered at a dose of about 50 mg/m², once every three weeks, and cisplatin that is administered at a dose of about 60 mg/m², once every three weeks, and capecitabine that is administered at a dose of about 1250 mg/m², daily.

In certain embodiments with that dosage of anti-HGF antibody and frequency of administration, for each administration, the administration of the at least one other therapeutic agent will be administered prior to the administration of the antibody to HGF.

In certain embodiments with that dosage of anti-HGF antibody and frequency of administration, for each administration, the administration of the at least one other therapeutic agent will be administered after the administration of the antibody to HGF. In certain embodiments with that dosage of anti-HGF antibody and frequency of administration, for each administration, the administration of the at least one other therapeutic agent will be administered at the same time as the administration of the antibody to HGF.

In certain embodiments, the frequency of dosing will take into account the pharmacokinetic parameters of the anti-HGF antibody and the at least one therapeutic agent, if used, in the formulation. In certain embodiments, the clinician may administer a therapeutically effective dose of an anti-HGF antibody and the therapeutic agent, if used, until the desired effect is achieved. In certain embodiments, a therapeutically effective dose of an anti-HGF antibody and a therapeutically effective dose of at least one additional therapeutic agent, if used, may be administered as a single dose or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them.

In certain embodiments, a therapeutically effective dose of an anti-HGF antibody comprises an amount of an anti-HGF antibody that increases over the course of a patient treatment. In certain embodiments, a therapeutically effective dose of an anti-HGF antibody comprises an amount of an anti-HGF antibody that decreases over the course of a patient treatment.

In certain embodiments, the dosing regimen includes an administration of a therapeutically effective dose of an anti-HGF antibody every week. In certain embodiments, the dosing regimen includes an administration of a therapeutically effective dose of an anti-HGF antibody every two weeks. In certain embodiments, the dosing regimen includes an administration of a therapeutically effective dose of an anti-HGF antibody every three weeks. In certain embodiments, the dosing regimen includes an administration of a therapeutically effective dose of an anti-HGF antibody every four weeks. In certain embodiments, the dosing regimen includes an administration of a therapeutically effective dose of an anti-HGF antibody every six weeks.

In certain embodiments, the dosing regimen includes an administration of a therapeutically effective dose of an anti-HGF antibody every three weeks for a treatment period of 2 cycles (or 6 weeks). In certain embodiments, the dosing regimen includes an administration of a therapeutically effective dose of an anti-HGF antibody every three weeks for a treatment period of 4 cycles (or 12 weeks). In certain embodiments, the dosing regimen includes an administration of a therapeutically effective dose of an anti-HGF antibody every three weeks for a treatment period of 6 cycles (or 18 weeks). In certain embodiments, the dosing regimen includes an initial administration of a therapeutically effective dose of an anti-HGF antibody every three weeks for a treatment period of 7 cycles (or 21 weeks). In certain embodiments, the dosing regimen includes an initial administration of a therapeutically effective dose of an anti-HGF antibody every three weeks for a treatment period of 8 cycles (or 24 weeks). In certain embodiments, the dosing regimen includes an initial administration of a therapeutically effective dose of an anti-HGF antibody every three weeks for a treatment period of 9 cycles (or 27 weeks). In certain embodiments, the dosing regimen includes an initial administration of a therapeutically effective dose of an anti-HGF antibody every three weeks for a treatment period of 10 cycles (or 30 weeks).

In certain embodiments, the dosing regimen includes an administration of a therapeutically effective dose of an anti-HGF antibody and at least one additional therapeutic agent every three weeks for a treatment period of 2 cycles (or 6 weeks). In certain embodiments, the dosing regimen includes an administration of a therapeutically effective dose of an anti-HGF antibody and at least one additional therapeutic agent every three weeks for a treatment period of 4 cycles (or 12 weeks). In certain embodiments, the dosing regimen includes an administration of a therapeutically effective dose of an anti-HGF antibody and at least one additional therapeutic agent every three weeks for a treatment period of 6 cycles (or 18 weeks). In certain embodiments, the dosing regimen includes an initial administration of a therapeutically effective dose of an anti-HGF antibody in combination with at least one additional therapeutic agent every three weeks for a treatment period of 7 cycles (or 21 weeks). In certain embodiments, the dosing regimen includes an initial administration of a therapeutically effective dose of an anti-HGF antibody in combination with at least one additional therapeutic agent every three weeks for a treatment period of 8 cycles (or 24 weeks). In certain embodiments, the dosing regimen includes an initial administration of a therapeutically effective dose of an anti-HGF antibody in combination with at least one additional therapeutic agent every three weeks for a treatment period of 9 cycles (or 27 weeks). In certain embodiments, the dosing regimen includes an initial administration of a therapeutically effective dose of an anti-HGF antibody in combination with at least one additional therapeutic agent every three weeks for a treatment period of 10 cycles (or 30 weeks).

In certain embodiments, treatment with the other therapeutic agent or therapeutic agents is terminated after 6 cycles of treatment, but treatment with the anti-HGF antibody continues for a treatment period comprising 21 weeks, 6 months, one year, or more. In certain embodiments, treatment with the other therapeutic agent or therapeutic agents is terminated after 7 cycles of treatment, but treatment with the anti-HGF antibody continues for a treatment period comprising 6 months, one year, or more. In certain embodiments, treatment with the other therapeutic agent or therapeutic agents is terminated after 8 cycles of treatment, but treatment with the anti-HGF antibody continues for a treatment period comprising 7 months, one year, or more. In certain embodiments, treatment with the other therapeutic agent or therapeutic agents is terminated after 9 cycles of treatment, but treatment with the anti-HGF antibody continues for a treatment period comprising 8 months, one year, or more. In certain embodiments, treatment with the other therapeutic agent or therapeutic agents is terminated after 10 cycles of treatment, but treatment with the anti-HGF antibody continues for a treatment period comprising 9 months, one year, or more.

In certain embodiments, the same therapeutically effective dose of an HGF-antibody and at least one other therapeutic agent, if used, is administered at each dosing over the course of a treatment period. In certain embodiments, different therapeutically effective doses of an anti-HGF antibody and at least one other therapeutic agent, if used, are administered at each dosing over the course of a treatment period. In certain embodiments, the same therapeutically effective dose of an anti-HGF antibody and at least one other therapeutic agent, if used, is administered at certain dosing over the course of a treatment period and different therapeutically effective doses are administered at certain other dosing.

In certain embodiments, the route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions may be administered by bolus injection or continuously by infusion, or by implantation device.

In certain embodiments, intravenous administration occurs by infusion over a period of 1 to 10 hours. In certain embodiments, intravenous administration occurs by infusion over a period of 1 to 8 hours. In certain embodiments, intravenous administration occurs by infusion over a period of 2 to 7 hours. In certain embodiments, intravenous administration occurs by infusion over a period of 4 to 6 hours. In certain embodiments, intravenous administration occurs by infusion over a period of 2 to 3 hours. In certain embodiments, intravenous administration occurs by infusion over a period of 1 to 2 hours. In certain embodiments, intravenous administration occurs by infusion over a period of 0.5 to 1 hour. In certain embodiments, intravenous administration occurs by infusion over a period of 0.1 to 0.5 hours. The determination of certain appropriate infusion periods is within the skill of the art. In certain embodiments, the initial infusion is given over a period of 4 to 6 hours, with subsequent infusions delivered more quickly. In certain such embodiments, subsequent infusions are administered over a period of I to 6 hours.

In certain embodiments, the infusion time period for administering an antibody to HGF in a dose of 15 mg/kg is 60 minutes±15 minutes. In certain embodiments, if a dose of an antibody to HGF is well tolerated (i.e., without any serious infusion-related reactions), then subsequent IV infusions of an antibody to HGF may be administered in a time period of 30 minutes±15 minutes. In certain embodiments with that dosage of anti-HGF antibody and frequency of administration, and infusion time periods, for each administration, the administration of the other therapeutic agent or therapeutic agents will be administered prior to the administration of the antibody to HGF. In certain embodiments with that dosage of antibody, frequency of administration, and infusion time periods, for each administration, the administration of the other therapeutic agent or therapeutic agents will be administered after the administration of the antibody to HGF. In certain embodiments with that dosage of antibody, frequency of administration, and infusion time periods, for each administration, the administration of the other therapeutic agent or therapeutic agents will be administered at the same time as the administration of the antibody to HGF.

As shown in the data presented herein, patients having tumors that have the c-Met protein levels described above show enhancements in overall survival and in progression-free survival when administered an anti-HGF antibody, such as rilotumumab, and at least one therapeutic agent, such as epirubicin, cisplatin and capecitabine. By performing the disclosed method, the medical professional can provide a more efficacious treatment regimen to patients suffering from this condition.

### EXAMPLES

The following examples, including the experiments conducted and results achieved are provided for illustrative purpose only and are not to be construed as limiting upon the claims.

### Example 1

### Amgen Study Number 20060317 (“The '317 Study)

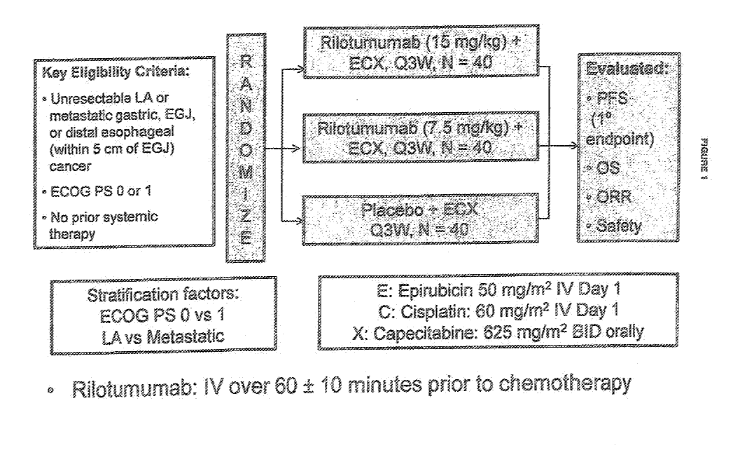

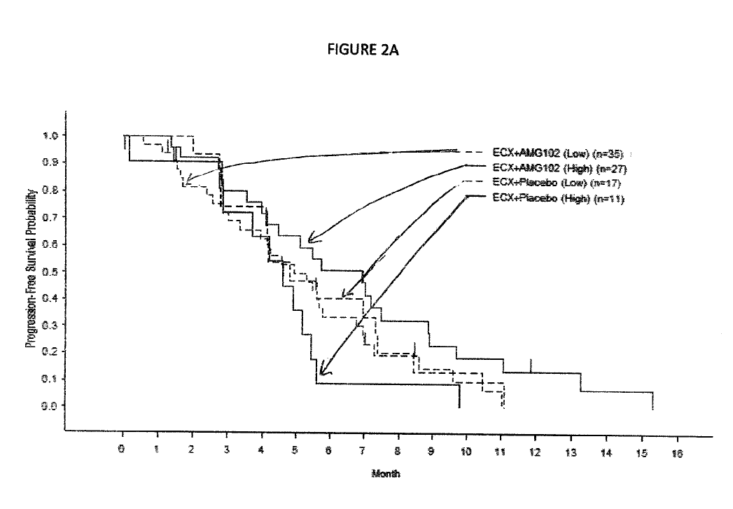

The '317 Study was a multicenter, double-blind, 3-arm, Phase 1b/2 Study in subjects with unresectable locally advanced or metastatic gastric or esophagogastric junction adenocarcinoma to evaluate the safety and efficacy of first-line treatment with epirubicin, cisplatin and capecitabine plus rilotumumab. Subjects were randomized in a 1:1:1 ratio to receive ECX+high dose AMG 102 (15 mg/kg) (Arm A), ECX+low dose AMG 102 (7.5 mg/kg)(Arm B) or ECX+placebo (Arm C). AMG 102 was administered as an IV infusion every 21 days (±3 days) along with ECX for up to ten cycles. ECX was administered as follows: Epirubicin (50 mg/m²) was administered as an IV bolus over 10+2 minutes and then cisplatin 60 mg/m²) was administered in normal saline over 4 hours (±15 minutes). Cistplatin could also be administered over 2 to 4 hours, according to local standard institutional practice. For preparation and complete prescribing information please refer to the most current package inserts in the region. Capecitabine tablets were available in 500 mg and 150 mg and was administered morning and evening and swallowed with water. The overall study design is described by a study schema in FIG. 1.

### Samples:

Archival tumor samples were available for 106 patients, 99 of which were acceptable for c-Met protein testing and 90 of which were from patients within the per protocol analysis set.

### PFS and OS:

PFS was the primary endpoint in the study. The goal of the primary analysis was to estimate the treatment effect on PFS of subjects with advanced gastric cancer receiving AMG 102 in combination with ECX compared to ECX/placebo. The timing of the primary analysis of PFS was event-driven based on the pre-specified goal for the target number of PFS events. Tumor response was assessed according to RECIST with modifications. Radiographical assessments were done by computed tomography (CT) or magnetic resonance imaging (MRI); the modality selected was the same for each subject throughout the study. Tumor response assessment was performed every 6 weeks starting from day 1, independent of treatment cycle, until documented disease progression (radiological or clinical), intolerable adverse event, withdrawal of consent or study discontinuation. The primary analysis of PFS and other efficacy endpoints was according to investigator assessment. OS was examined as a secondary endpoint.

### Immunohistochemistry:

C-Met protein was measured using Immunohistochemistry (“IHC”). IHC was performed in accordance with Mosaic Laboratories' Standard Operating Procedures and validated protocol. The c-Met immunohistochemistry (IHC) assay was designed and validated to be compatible with CLIA guidelines for “homebrew” class I test validation. The validated procedure for IHC analysis of c-Met was performed using manual detection at room temperature (RT). Samples to be stained were received as unstained slides (5-micron). All slides for staining were baked, deparaffinized, and rehydrated. Following rehydration, tissue sections were incubated in Envision peroxidase (Dako, Carpentaria, Calif.) for 5 minutes to quench endogenous peroxidase. Tissue sections then underwent pretreatment using Borg Buffer (Biocare Medical, Concord, Calif.) for 30 seconds in a decloaker set to 125° C. followed by a rinse in Splash-T Buffer (0.05%, Mosaic Laboratories, Lake Forest, Calif.). Slides were blocked with Animal-Free block (Dako) for 5 minutes at RT and tapped off. Slides were incubated with anti-c-Met antibody (R&D Systems, AF276) diluted in diluent (Dako) for 30 minutes. Slides were then rinsed twice in buffer for 5 minutes each followed by detection using rabbit-anti goat antibody (Vector Labs, Burlingame, Calif.) for 15 minutes. Slides were then rinsed twice in buffer for 5 minutes each followed by detection using the Envision+Rabbit HRP detection kit (Dako) for 15 minutes. Slides were rinsed twice with buffer for 5 minutes each followed by incubation with DAB (Dako) for 5 minutes. Slides were rinsed with water, counterstained with hematoxylin (Dako), blued in ammonia water, dehydrated through graded alcohols, cleared in xylene, and coverslipped.

### Data Analysis:

The study pathologist reviewed each sample for the presence of staining in the cytoplasm and membrane of tumor cells, which was recorded separately. The total percent positive staining in the tumor was also recorded along with the maximum staining intensity observed in the normal adjacent tissue (NAT), endothelia, smooth muscle, fibroblasts, stroma, and nerve, where applicable. The c-Met assay was evaluated on a semi-quantitative scale, and the percentage of cancer cells staining at each of the following four levels was recorded: 0 (unstained), 1+(weak staining), 2+(moderate staining) and 3+(strong staining). An H-score was calculated based on the

summation of the product of percent of cells stained at each intensity using the following equation: (3×% cells staining at 3+)+(2×% cells staining at 2+)+(1×% cells staining at 1+).

All following analyses were based on a subset of subjects in the Per Protocol Analysis Set with archival tumor sample and measurable c-Met by IHC. The “Per Protocol Analysis Set” is an analysis set that includes all randomized subjects who had received at least one dose of rilotumumab and without pre-specified important protocol deviations (including those subjects not receiving rilotumumab) that may have potentially impacted estimation of efficacy endpoints. Subjects were analyzed according to the actual treatment received.

### A. C-Met IHC Cytoplasmic Percent Positive >50% (High) Versus Cytoplasmic Percent Positive <50% (Low):

Exploratory findings of treatment effect within patients dichotomized by c-Met expression by IHC (high vs. low expression groups) were reported. Median of c-Met IHC cytoplasmic percent positive was 50%. Dichotomization was defined as c-Met IHC cytoplasmic percent positive cells >50% (High) versus cytoplasmic percent positive <=50% (Low).

A Cox regression model adjusted by the stratification factors was used to estimate the adjusted Progression Free Survival (“PFS”) (or Overall Survival (“OS”)), hazard ratio (HR) and 95% confidence interval (CI) for both the rilotumumab arms combined vs. the placebo arm of patients within the high and the low expression groups, respectively. Stratification factors included locally advanced versus metastatic disease and Eastern Cooperative Oncology Group (“ECOG”) performance status 0 versus 1. Interaction p-value for testing the heterogeneity of the treatment effect between the high and low expression groups was determined. Kaplan-Meier (K-M) curves were prepared for patients within the high vs. low expression groups in rilotumumab arms combined or placebo arm. For PFS, the resulted HRs and CIs for rilotumumab arms combined vs. placebo were: HR=1.014 with 95% CI=(0.533, 1.931) in low expression group, whereas HR=0.526 with 95% CI=(0.245, 1.126) in high expression group with the corresponding interaction p-value of 0.093. For OS, the resulting HRs and CIs for rilotumumab arms combined vs. placebo were: HR=1.838 with 95% CI=(0.778, 4.343) in low expression group, whereas HR=0.290 with 95% CI=(0.111, 0.760) in high expression group with the interaction p-value of 0.007.

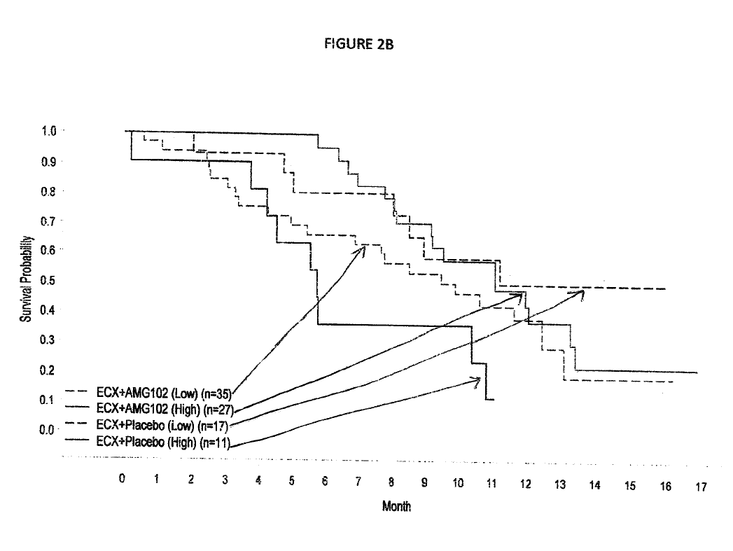

The median K-M estimates and 80% CI for the progression-free survival time (months) were: 5.3 (4.2, 5.7), 6.9 (5.1, 7.5), 4.8 (4.1, 7.0), and 4.6 (3.7, 5.2) of patients within the low and high expression subgroup within rilotumumab arms combined, and low and high expression subgroup within placebo arm, respectively. The Kaplan-Meier plots for PFS are shown in FIG. 2A.

The median K-M estimates and 80% CI for the overall survival time (months) were: 9.9 (7.7, 11.6), 11.1 (9.2, 13.3), NE (8.5, NE), and 5.7 (4.5, 10.4) in low and high biomarker subgroup within rilotumumab arms combined, and low and high biomarker subgroup within placebo arm, respectively. The Kaplan-Meier plots for OS are shown in FIG. 2B.

### B. C-Met IHC Cytoplasmic Percent Positive >10% (High) Versus Cytoplasmic Percent Positive <10% (Low):

Exploratory findings of the treatment effect within patients dichotomized by tumor c-Met expression (high vs. low expression groups) were reported. The 1st quartile of c-Met IHC Cytoplasmic percent positive was 10%. Dichotomization was defined as c-Met IHC Cytoplasmic percent positive cells >10% (High) vs. Cytoplasmic percent positive <=10% (Low).

A Cox regression model adjusted by the stratification factors was used to estimate the adjusted PFS (or OS) hazard ratio (HR) and 95% confidence interval (CI) of patients in both the rilotumumab arms combined vs. the placebo arm within the high and the low expression groups, respectively. Stratification factors included locally advanced versus metastatic disease and ECOG performance status 0 versus 1. Interaction p-value for testing the heterogeneity of the treatment effect between the high and low expression groups was determined. Kaplan-Meier (K-M) curves were prepared for patients in the high vs. low expression groups in rilotumumab arms combined or placebo arm. For PFS, the resulting HRs and CIs for rilotumumab arms combined vs. placebo were: HR=0.897 with 95% CI=(0.332, 2.422) in low expression group, whereas HR=0.658 with 95% CI=(0.372, 1.163) in high expression group with the corresponding interaction p-value of 0.513. For OS, the resulting HRs and CIs for rilotumumab arms combined vs. placebo were: HR=1.469 with 95% CI=(0.406, 5.313) in low expression group whereas HR=0.847 with 95% CI=(0.429, 1.672) in high expression group with the interaction p-value of 0.563.

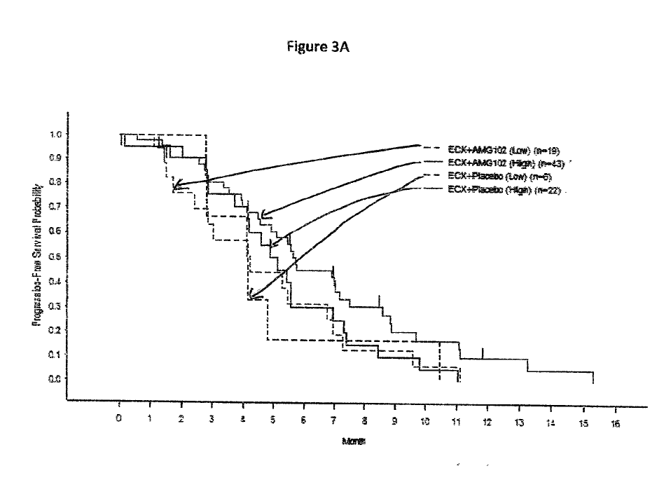

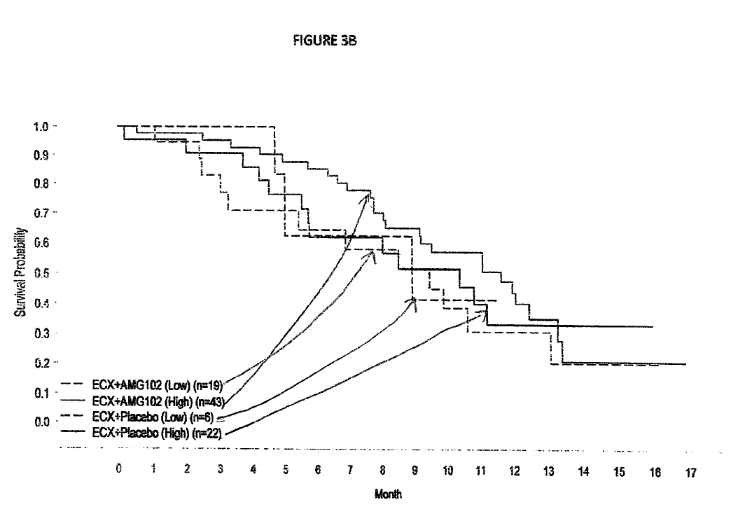

The median Kaplan-Meier estimates and 80% CI for the progression-free survival time (months) were: 4.2 (2.9, 5.5), 5.7 (5.1, 7.0), 4.1 (2.8, 4.8), and 5.2 (4.2, 5.6) in low and high expression subgroup within rilotumumab arms combined, and low and high expression subgroup within placebo arm, respectively. The Kaplan-Meier plots for PFS are shown in FIG. 3A.

The median K-M estimates and 80% CI for the overall survival time (months) were: 9.5 (5.4, 10.6), 11.6 (9.2, 12.5), 8.9 (5.0, NE), and 10.4 (5.7, 11.2) for patients in the low and high biomarker subgroup within rilotumumab arms combined, and low and high biomarker subgroup within placebo arm, respectively. The Kaplan-Meier plots for OS are shown in FIG. 3B.

### C. C-Met IHC Cytoplasmic Percent Positive >80% (High) Versus Cytoplasmic Percent Positive <80% (Low):

Exploratory findings of the treatment effect within patients dichotomized by tumor c-Met expression (high vs. low expression groups) were reported. The 3rd quartile of c-Met IHC Cytoplasmic percent positive was 80%. Dichotomization was defined as c-Met IHC Cytoplasmic percent positive cells >80% (High) vs. Cytoplasmic percent positive ≦80% (Low).

A Cox regression model stratified by the stratification factors was used to estimate the adjusted PFS (or OS) hazard ratio (HR) and 95% confidence interval (CI) of patients within both the rilotumumab arms combined vs. the placebo arm within the high and the low expression groups, respectively. Stratification factors included locally advanced versus metastatic disease and ECOG performance status 0 versus 1. Interaction p-value for testing the heterogeneity of the treatment effect between the high and low expression groups was determined. Kaplan-Meier (K-M) curves were prepared for patients in the high vs. low expression groups in rilotumumab arms combined or placebo arm. For PFS, the resulted HRs and Cis for rilotumumab arms combined vs. placebo were: HR=0.813 with 95% CI=(0.467, 1.414) in low expression group, whereas HR=0.668 with 95% CI=(0.204, 2.184) in high expression group with the corresponding interaction p-value of 0.170. For OS, the resulting HRs and CIs for rilotumumab arms combined vs. placebo were: HR=1.473 with 95% CI=(0.700, 3.102) in low expression group, whereas HR=0.166 with 95% CI=(0.033, 0.823) in high expression group with the interaction p-value of 0.010.

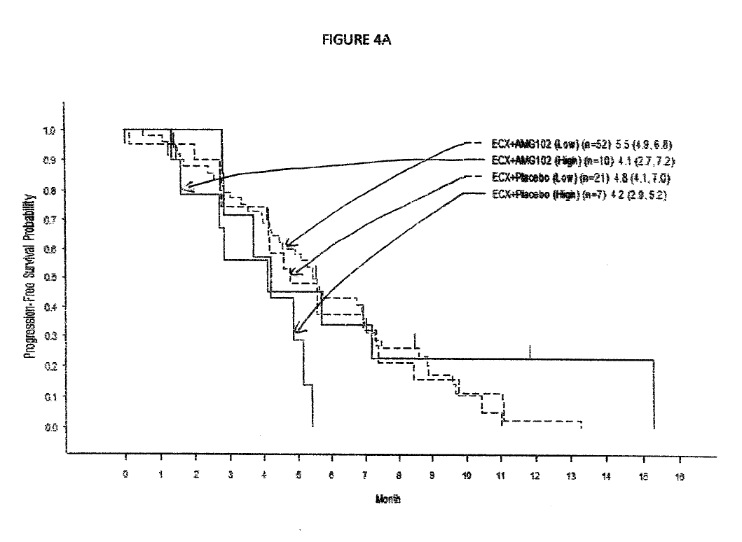

The median K-M estimates and 80% CI for the progression-free survival time (months) were: 5.5 (4.9, 6.8), 4.1 (2.7, 7.2), 4.8 (4.1, 7.0), and 4.2 (2.9, 5.2) in low and high expression subgroup within rilotumumab arms combined, and low and high expression subgroup within placebo arm, respectively. The Kaplan-Meier plots for PFS are shown in FIG. 4A.

The median Kaplan-Meier estimates and 80% CI for the overall survival time (months) were: 10.6 (8.5, 12.0), 11.1 (8.1, NE), 11.2 (8.5, NE), and 5.5 (4.2, 10.4) in low and high biomarker subgroup within rilotumumab arms combined, and low and high biomarker subgroup within placebo arm, respectively. The Kaplan-Meier plots for OS are shown in FIG. 4B.

D. Treatment Effect in Patients within High/Low c-Met IHC Subgroups Based on Various Cytoplasmic Percent Positive Specimens:

Exploratory findings of treatment effect (Treatment vs. Placebo) within patients dichotomized by tumor c-Met expression (high vs. low expression groups) was evaluated.

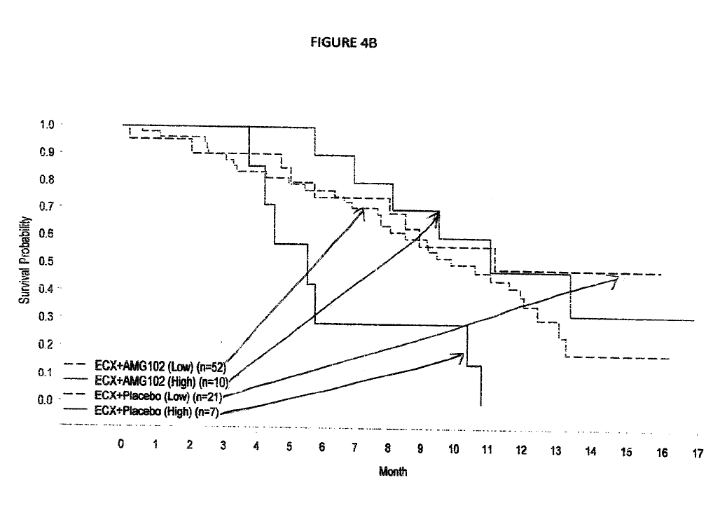

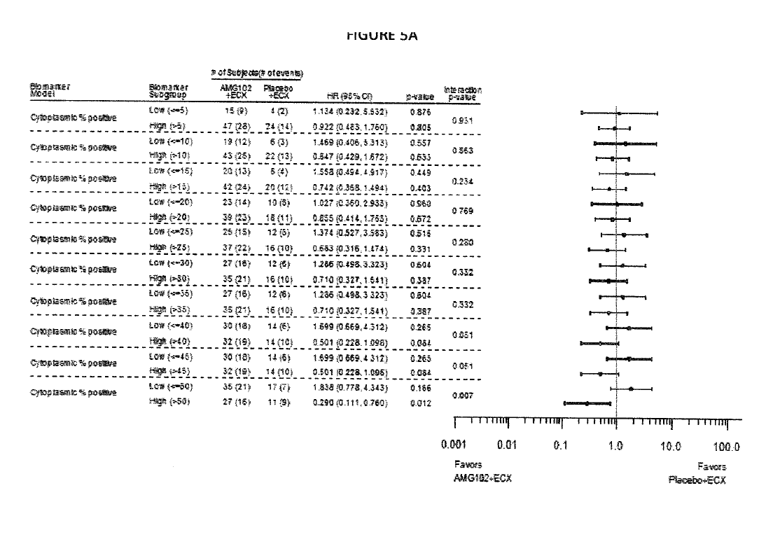

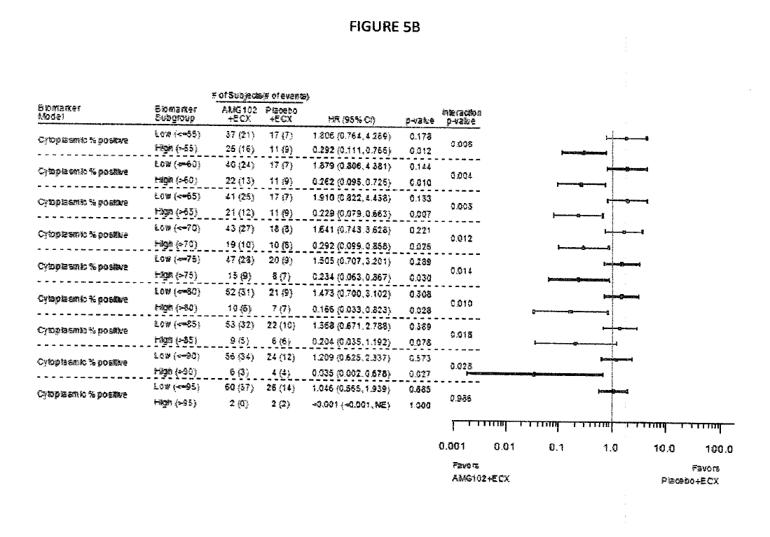

Interaction p-value for testing the heterogeneity of the treatment effect between the high and low expression groups was presented. Various ways of cutoff defining high and low expression group based on cytoplasmic percent positive were explored. Cox regression model stratified by the stratification factors was used to estimate the adjusted OS hazard ratio (HR) and 95% confidence interval (CI) for patients in both the rilotumumab arms combined vs. the placebo arm within the high and the low expression groups, respectively. Stratification factors included locally advanced versus metastatic disease and ECOG performance status 0 versus 1. FIG. 5 shows a forestplot summarizing the treatment effect in patients in the high/low c-Met IHC groups based on various cutoff of Cytoplasmic percent positive.

E. Treatment Effect in Patients within High/Low c-Met IHC Subgroups Based on Membrane, Cytoplasmic and Total Staining:

Exploratory findings of the treatment effect within patients with different dichotomized tumor c-Met expression (high vs. low expression groups) was evaluated. In this analysis several dichotomizations were used to define patients within whose tumors were in the c-Met IHC low vs. high expression groups: each dichotomization applied to both membrane and cytoplasmic staining separately and together (Total Staining). The dichotomizations were:

1) Low: Max SI<2+vs. High: Max SI≧2+:
2) Low H score vs. high H score (cut point is defined by 50% of the sample results);
3) Low percent positive vs. High percent positive (cut point is defined by 50% of sample results); and
4) Low Percent Positive 0-50% vs. High Percent positive 50-100%

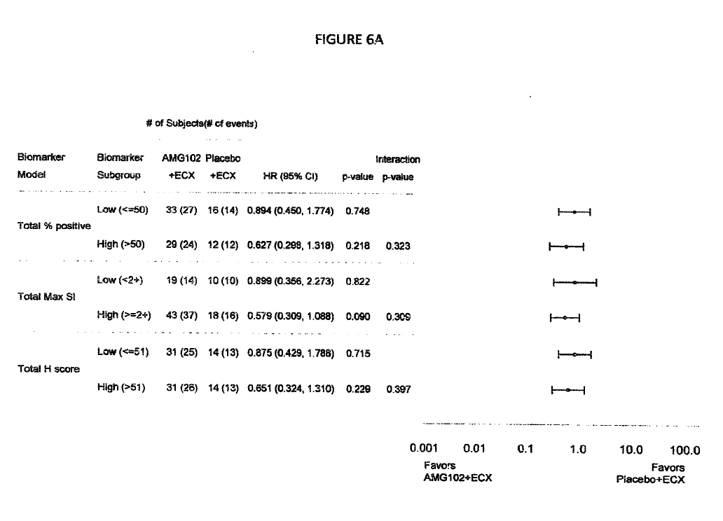

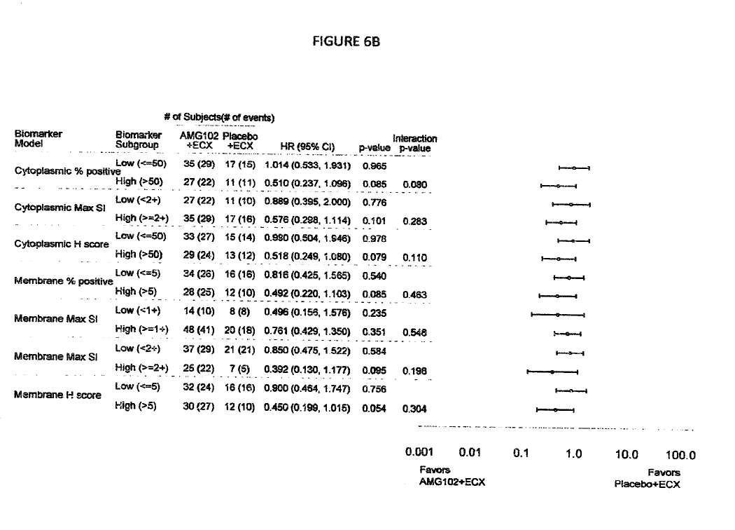

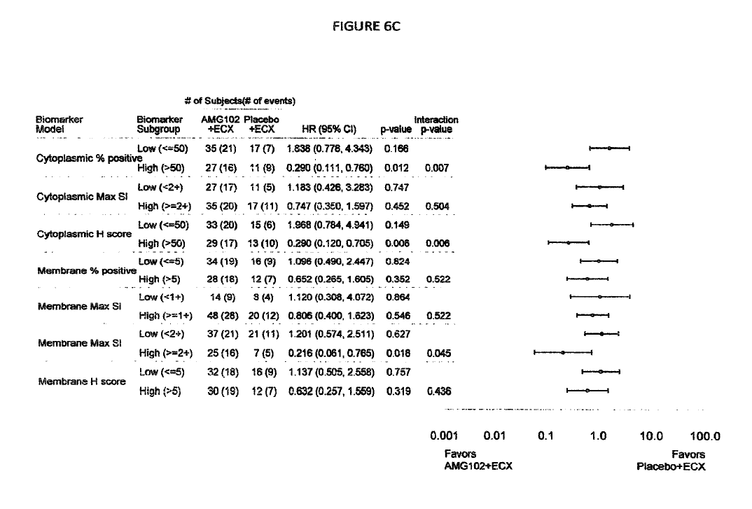

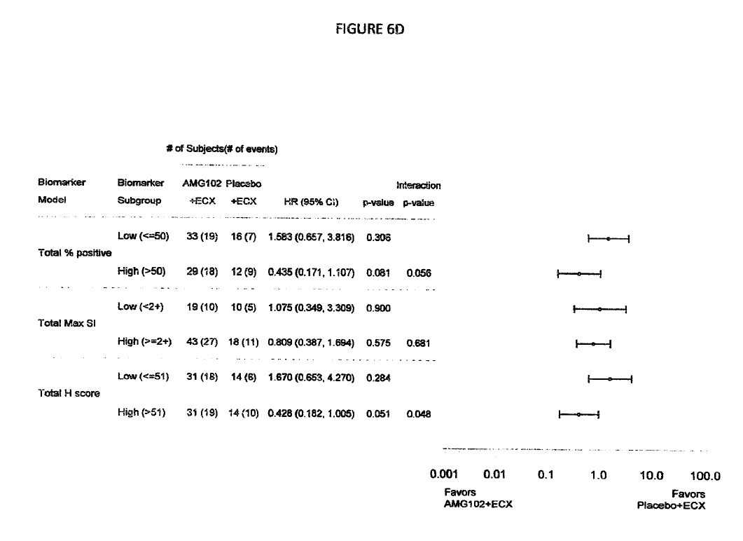

Cox regression model adjusted by the stratification factors was used to estimate the adjusted PFS (or OS) hazard ratio (HR) and 95%/0 confidence interval (CI) for patients within both the rilotumumab arms combined vs. the placebo arm within the high and the low expression groups, respectively. Stratification factors included locally advanced versus metastatic disease and ECOG performance status 0 versus 1. FIGS. 6A-D show forestplots summarizing the treatment effect in patients within the high/low c-Met IHC group based on various ways of c-Met IHC subgroups based on Membrane, Cytoplasmic and Total staining data.

### F: C-Met IHC Cytoplasmic H-Score Versus Cytoplasmic Percent Positive:

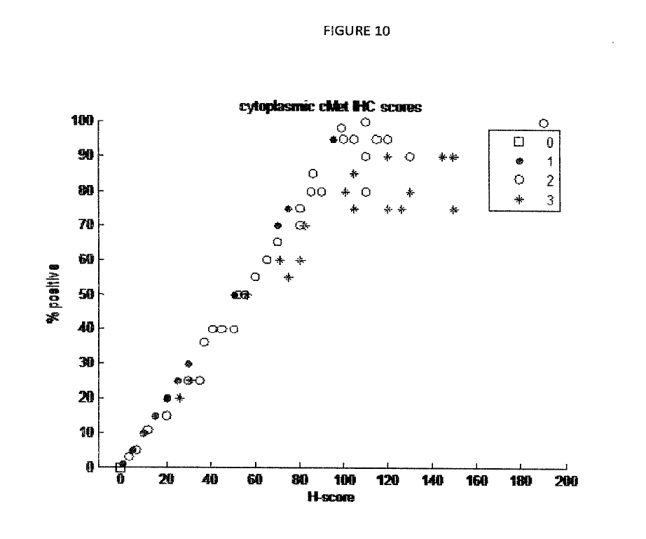

In this analysis, cytoplasmic c-Met protein levels were measured in tumor samples using IHC and expressed in H-Score, Maximum Staining Intensity (“MSI”) and percent positive. FIG. 10 is a scatter plot of cytoplasmic c-Met H-Score versus cytoplasmic c-Met percent positive. FIG. 10 shows the correlation of the various methods of expressing cytoplasmic c-Met.

Other embodiments will be apparent to those skilled in the art from consideration of the specification and practice of the invention disclosed herein. It is intended that the specification and examples be considered as exemplary only.

1. A method for predicting the efficacy of an anti-HGF antibody, comprising the step of determining a percentage of tumor cells having c-Met protein in a sample obtained from a patient diagnosed with gastric cancer, wherein a percentage of at least 1 percent of the tumor cells having c-Met predicts that administration of the anti-HGF antibody will treat the gastric cancer in the patient.

2. A method of predicting whether a patient suffering from gastric cancer will respond to treatment with an anti-HGF antibody, comprising the step of determining a percentage of tumor cells having c-Met protein in a sample obtained from a patient diagnosed with gastric cancer, wherein a percentage of at least 1 percent of tumor cells having c-Met protein predicts that administration of the anti-HGF antibody will treat the gastric cancer in the patient.

3. A method of screening for patients diagnosed with gastric cancer as being responsive to treatment with an anti-HGF antibody comprising the step of determining a percentage of tumor cells having c-Met protein present in a sample obtained from a patient diagnosed with gastric cancer, wherein a percentage of at least 1 percent of tumor cells having c-Met protein predicts that the patient with gastric cancer will be responsive to treatment with an anti-HGF antibody.

4. The method according to claim 1, wherein at least 25 percent of the tumor cells have c-Met protein present.

5. The method according to claim 4, wherein at least 50 percent of the tumor cells have c-Met protein present.

6. The method according to claim 1, wherein the c-Met protein is present predominantly in the cytoplasm of the tumor cells.

7. The method according to claim 1, wherein the c-Met protein is present predominantly in the membrane of the tumor cells.

8. A method for predicting the efficacy of an anti-HGF antibody, comprising the step of determining the maximum staining intensity of c-Met protein in tumor cells obtained from a patient diagnosed with gastric cancer, wherein a maximum staining intensity of at least 1 predicts that administration of the anti-HGF antibody will treat the gastric cancer in the patient when administered.

9. A method of predicting whether a patient suffering from gastric cancer will respond to treatment with an anti-HGF antibody, comprising the step of determining the maximum staining intensity of c-Met protein in tumor cells obtained from a patient diagnosed with gastric cancer, wherein a maximum staining intensity of at least 1 predicts that administration of the anti-HGF antibody will treat the gastric cancer in the patient.

10. A method of screening for patients diagnosed with gastric cancer as being responsive to treatment with an anti-HGF antibody comprising the step of determining the maximum staining intensity of c-Met protein in tumor cells obtained from a patient diagnosed with gastric cancer, wherein a maximum staining intensity of at least 1 predicts that the patient with gastric cancer will be responsive to treatment with an anti-HGF antibody.

11. The method according to claim 8, wherein the maximum staining intensity is at least 2.

12. The method according to claim 8, wherein the maximum staining intensity is at least 3.

13. The method according to claim 8, wherein the c-Met protein is present predominantly in the cytoplasm of the tumor cells.

14. The method according to claim 8, wherein the c-Met protein is present predominantly in the membrane of the tumor cells.

15. A method for predicting the efficacy of an anti-HGF antibody, comprising the step of determining the H-score for c-Met protein in tumor cells obtained from a patient diagnosed with gastric cancer, wherein an H-score of at least 1 predicts that administration of the anti-HGF antibody will treat the gastric cancer in the patient.

16. A method of predicting whether a patient suffering from gastric cancer will respond to treatment with an anti-HGF antibody, comprising the step of determining the H-score for c-Met protein in tumor cells obtained from a patient diagnosed with gastric cancer, wherein an H-score for c-Met protein of greater than 1 predicts that administration of the anti-HGF antibody will treat the gastric cancer in the patient.

17. A method of screening for patients diagnosed with gastric cancer as being responsive to treatment with an anti-HGF antibody comprising the step of determining the H-score of c-Met protein in tumor cells obtained from a patient diagnosed with gastric cancer, wherein an H-score for c-Met protein of at least 1 predicts that the patient with gastric cancer will be responsive to treatment with an anti-HGF antibody.

18. The method according to claim 15, wherein the H-score is greater than 50.

19. The method according to claim 15, wherein the H-score is greater than 100.

20. The method according to claim 15, wherein the H-score is greater than 200.

21. The method according to claim 15, wherein the c-Met protein is present predominantly in the cytoplasm of the tumor cells.

22. The method according to claim 15, wherein the c-Met protein is present predominantly in the membrane of the tumor cells.

23. A method of treating a patient diagnosed with gastric cancer, wherein a sample of tumor cells obtained from the patient diagnosed with gastric cancer has a percentage of at least 1 percent of the tumor cells having c-Met protein present, as measured in an in vitro assay, the method comprising the step of administering to a patient diagnosed with gastric cancer an anti-HGF antibody effective to provide a therapeutic benefit.

24. The method according to claim 23, wherein at least 25 percent of the tumor cells have c-Met protein present.

25. The method according to claim 23, wherein at least 50 percent of the tumor cells have c-Met protein present.

26. The method according to claim 23 wherein at least 75 percent of the tumor cells have c-Met protein present.

27. The method according to claim 23, wherein the c-Met protein is measured in the cytoplasm of the tumor cells.

28. The method according to claim 23, wherein the c-Met protein is measured in the membrane of the tumor cells.

29. The method according to claim 28, wherein the c-Met protein is further measured in the cytoplasm of the tumor cells.

30. A method of treating a patient diagnosed with gastric cancer, wherein a sample of tumor cells obtained from the patient diagnosed with gastric cancer has a maximum staining intensity of c-Met protein in tumor cells of at least 1, as measured in an in vitro assay, the method comprising the step of administering to a patient diagnosed with gastric cancer an anti-HGF antibody effective to provide a therapeutic benefit.

31. The method according to claim 30, wherein the maximum staining intensity is at least 2.

32. The method according to claim 30, wherein the maximum staining intensity is at least 3.

33. The method according to claim 30, wherein the c-Met protein is measured in the cytoplasm of the tumor cells.

34. The method according to claim 30, wherein the c-Met protein is measured in the membrane of the tumor cells.

35. The method according to claim 34, wherein the c-Met protein is further measured in the cytoplasm of the tumor cells.

36. A method of treating a patient diagnosed with gastric cancer, wherein a sample of tumor cells obtained from the patient diagnosed with gastric cancer has an H-score for c-Met protein of at least 1, as measured in an in vitro assay, the method comprising the step of administering to a patient diagnosed with gastric cancer an anti-HGF antibody effective to provide a therapeutic benefit.

37. The method according to claim 36, wherein the H-score is greater than 50.

38. The method according to claim 36, wherein the H-score is greater than 100.

39. The method according to claim 36, wherein the H-score is greater than 200.

40. The method according to claim 36, wherein the c-Met protein is measured in the cytoplasm of the tumor cells.

41. The method according to claim 36, wherein the c-Met protein is measured in the membrane of the tumor cells.

42. The method according to claim 41, wherein the c-Met protein is further measured in the cytoplasm of the tumor cells.

43-62. (canceled)
